(19) United States
(12) Patent Application Publication
(10) Pub. No.: US 2025/0374926 A1
(43) Pub. Date: Dec. 11, 2025

(54) NOVEL DERIVATIVES OF NON-CODED AMINO ACIDS AND THEIR USE IN CROP PROTECTION

(71) Applicant: FORTEPHEST LTD., Ness Ziona (IL)

(72) Inventors: Alex Kozak, Ness Ziona (IL); Israel Shapiro, Rehovot (IL); Marina Zholkovsky, Bat-Yam (IL); Erel Hatan, Kfar-HaNagid (IL); Alon Glick, Mazkeret Batia (IL)

(73) Assignee: FORTEPHEST LTD., Ness Ziona (IL)

(21) Appl. No.: 18/876,582

(22) PCT Filed: Jun. 26, 2023
(86) PCT No.: PCT/IL2023/050657

§ 371 Date: Dec. 18, 2024

Related U.S. Application Data
(60) Provisional application No. 63/367,074, filed on Jun. 27, 2022.
(60) Provisional application No. 63/369,396, filed on Jul. 26, 2022.

(51) Int. Cl. A01N 57/16; A01N 37/02; A01N 37/10; A01N 43/40; A01N 43/78; A01N 47/06; A01P 13/02; C07D 213/30; C07D 213/32; C07D 277/34; C07D 277/36; C07F 9/58; C07F 9/6539

(52) U.S. Cl. CPC A01N 57/16; A01N 37/02; A01N 37/10; A01N 43/40; A01N 43/78; A01N 47/06; A01P 13/02; C07D 213/30; C07D 213/32; C07D 277/34; C07D 277/36; C07F 9/58; C07F 9/6539

(57) ABSTRACT

Novel herbicidal compositions and their applications in crop protection, specifically in controlling growth of harmful weeds are provided.

### FIELD OF THE INVENTION

The invention relates to novel chemical compounds having herbicidal activity, process for their manufacture and their use in crop protection.

### BACKGROUND OF THE INVENTION

Besides their role as protein constituents, amino acids and their derivatives are also involved in a plethora of cellular reactions, and therefore, they influence several physiological processes such as plant growth and development, intracellular pH control, generation of metabolic energy or redox power, and resistance to both abiotic and biotic stress. Furthermore, amino acids role during signaling in plants has recently been discussed. In this context, regulation of amino acid content, fluxes, and transport through the plant are critical for plant adaptation to carbon and nitrogen status, development, and defense.

Pools of all amino acids are much induced during stress. This implies that selective amino acids and their metabolites could be considered as a scaffold for the development of new plant-protecting products. Proline levels are known to significantly increase, as part of the stress response in several plants and considered to represent a compatible osmolyte. Moreover, branched-chain amino acids are also much induced during various stresses.

Mechanisms underlying regulation of amino acids pools require further elucidations. Local amino acid concentrations depend on the synthesis and degradation rates of proteins as well as amino acids and on transport processes. These processes rather modify the total pool size, which is especially relevant when bulk degradation takes place such as during germination and senescence.

New compounds effective for controlling the growth of undesired vegetation are in constant demand. In the most common situation, such compounds are sought to selectively control the growth of weeds in useful crops such as cotton, rice, corn/maze, wheat and soybeans, to name a few. Unchecked weed growth in such crops can cause significant losses, reducing profit for the farmer and increasing costs to the consumer. In other situations, herbicides are desired to control all plant growth. Examples of areas in which complete control of all vegetation is desired are areas around railroad tracks, storage tanks, and industrial storage areas. Identifying new compounds having herbicidal activity, which are more effective, less costly and environmentally safe remains long and unmet need.

### SUMMARY OF THE INVENTION

It is a principal object of the present invention to provide novel, safe and potent herbicides.

According to some embodiments, the invention provides an herbicidal composition comprising a compound or a salt thereof having the structure

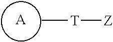

or a salt thereof, wherein:
A is a cyclopentadiene, benzene and indene scaffold comprising from 1 to 4 heteroatoms; wherein each of the heteroatoms is independently selected from the group consisting of N, S, Se, and O; and wherein one or more of the carbon, nitrogen, sulfur or selenium atoms of the ring are optionally chemically attached to at least one of the groups consisting of: —CX₃, —CX₂R,—COX, —CHO, —COR,-CO₂R, —CONH₂, —CONHR, sulfur ═S, oxygen ═0, —F, —Cl, —Br, and —I, and wherein X is selected from F, Cl, Br and I; and
T is —(CH₂)ₙ—, —OR₁, —SR₁, —SOR₁, —CSR₁, —CS—, —POHR₁, —PO(OR₁)R₁, —CO—, —C(O)R₁, and —COOR₁;
Z is H, substituted or non-substituted alkyl, substituted or non-substituted aryl group, —COOH, —COO—, —OH, —O—R, —COOR with saturated or non-saturated alcohol residues with straight, branched, cyclic, aromatic or heteroaromatic chain; —O—(CH₂CH₂O)ₙR (n≥1); —O—(CHMeCH₂O)ₙR (n≥1)); substituted or unsubstituted phosphate group, sulfonyl group, —SO₃H, sulfo group, sulfonic/sulphonic acid, cyano carbamoyl group, primary amine, secondary amine, tertiary amine, carboxamide, —NR—O—R, —O—NR, or a salt thereof; and
   wherein R is H, substituted or non-substituted alkyl, and substituted or non-substituted aryl group; and R₁ is —(CH₂)ₙ— (n is 0 to 9); and, wherein the composition comprises at least one agriculturally acceptable carrier.

According to some embodiments, the invention further provides the herbicidal composition, according to the above, wherein Z is selected from

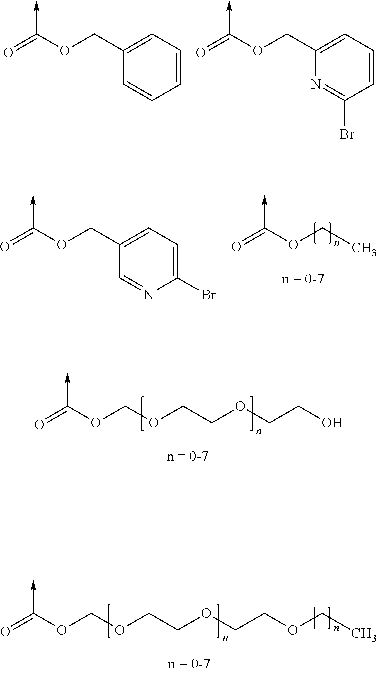

According to some embodiments, the invention further provides the herbicidal composition, according to the above, comprising the compound having the structure

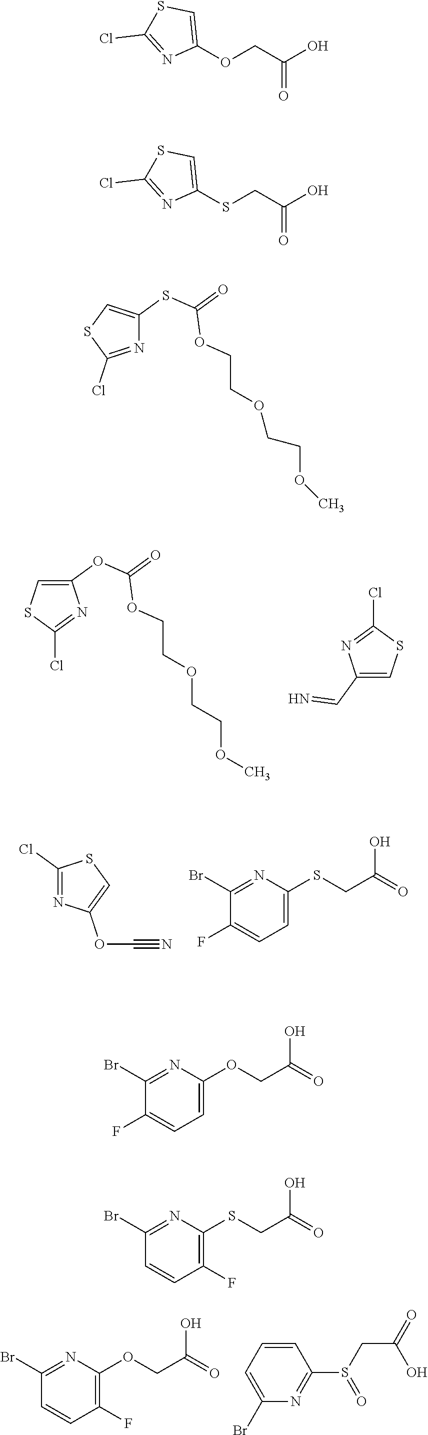

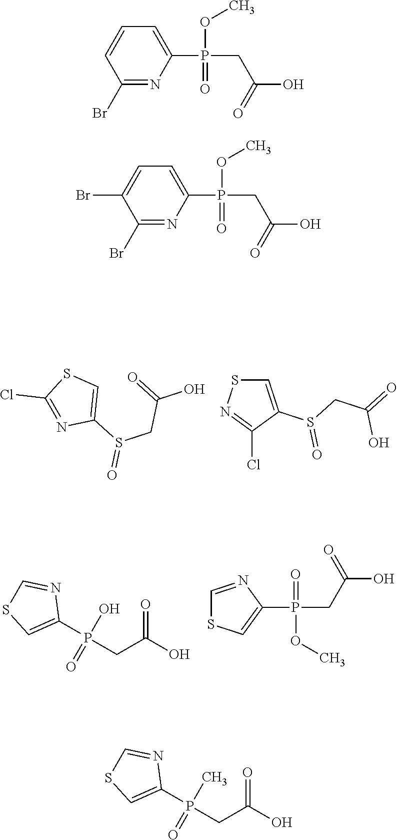

According to some embodiments, the invention provides an herbicidal compound having the structure selected from

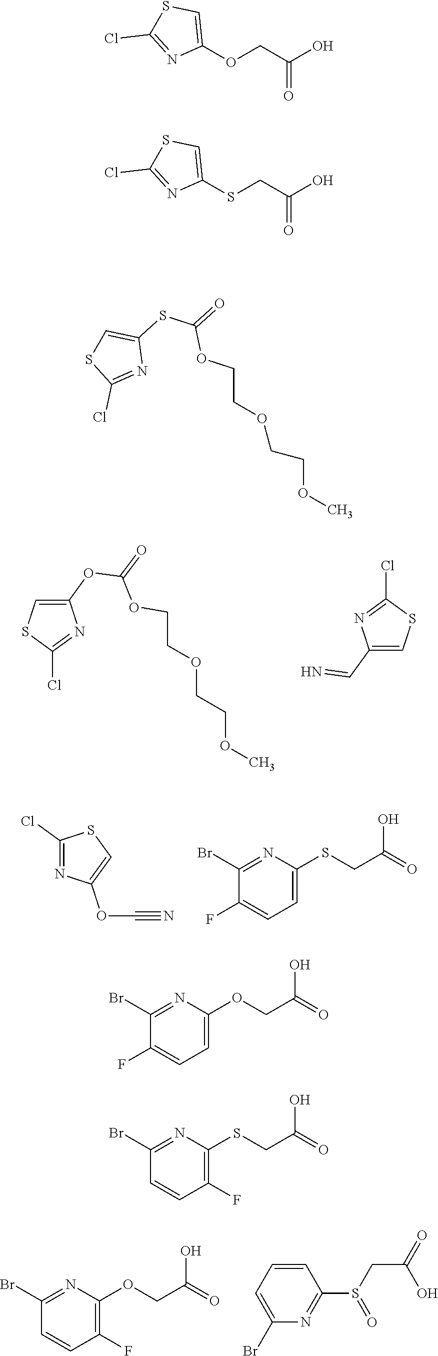

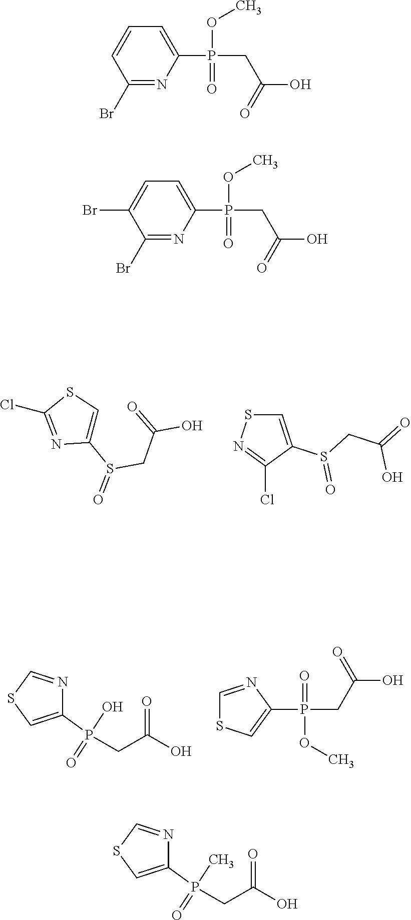

or a salt thereof.

According to some embodiments, the invention provides an herbicidal composition for controlling undesired plant growth comprising a mixture of:
a. a compound having the structure

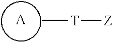

or a salt thereof, wherein:
   A is a cyclopentadiene, benzene and indene scaffold comprising from 1 to 4 heteroatoms; wherein each of the heteroatoms is independently selected from the group consisting of N, S, Se, and O; and wherein one or more of the carbon, nitrogen, sulfur or selenium atoms of the ring are optionally chemically attached to at least one of the groups consisting of:-CX₃, —CX₂R, —COX, —CHO, —COR, —CO₂R, —CONH₂, —CONHR, sulfur ═S, oxygen ═O, —F, —Cl, —Br, and —I, and wherein X is selected from F, Cl, Br and I;
   T is —(CH₂)ₙ—, —OR₁, —SR₁, —SOR₁, —CSR₁, —CS—, —POHR₁, —PO(OR₁)R₁, —CO—, —COR₁, and —COOR₁;
   Z is H, substituted or non-substituted alkyl, substituted or non-substituted aryl group, —COOH, COO—, —OH, —O—R, —COOR with saturated or non-saturated alcohol residues with straight, branched, cyclic, aromatic or heteroaromatic chain; —O—(CH₂CH₂O)ₙR (n≥1);—O—(CHMeCH₂O)ₙR (n≥1)); substituted or unsubstituted phosphate group, sulfonyl group, sulfo group, sulfonic/sulphonic acid, —SO₃H, carbamoyl group, primary amine, secondary amine, tertiary amine, carboxamide, cyano group, —NR—O—R, —O—NR, or a salt thereof; and
   wherein R is H, substituted or non-substituted alkyl, and substituted or non-substituted aryl group; and, R₁ is —(CH₂)ₙ— (n is 0 to 9);
b. at least one plant growth regulator; and
c. at least one agriculturally acceptable carrier.

According to some embodiments, the invention provides an herbicidal composition for controlling undesired plant growth comprising a mixture of:
a. a compound having the structure

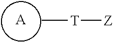

or a salt thereof, wherein:
   A is a cyclopentadiene, benzene and indene scaffold comprising from 1 to 4 heteroatoms; wherein each of the heteroatoms is independently selected from the group consisting of N, S, Se, and O; and wherein one or more of the carbon, nitrogen, sulfur or selenium atoms of the ring are optionally chemically attached to at least one of the groups consisting of: —CX₃, —CX₂R, —COX, —CHO, —COR, —CO₂R, —CONH₂, —CONHR, sulfur ═S, oxygen ═O, —F, —Cl, —Br, and —I, and wherein X is selected from F, Cl, Br and I;
   T is —(CH₂)ₙ—, —OR₁, —SR₁, —SOR₁, —CSR₁, —CS—, —POHR₁, —PO(OR₁)R₁, —CO—, —COR₁, and —COOR₁;
   Z is H, substituted or non-substituted alkyl, substituted or non-substituted aryl group, —COOH, COO—, —OH, —O—R, —COOR with saturated or non-saturated alcohol residues with straight, branched, cyclic, aromatic or heteroaromatic chain; —O—(CH₂CH₂O)ₙR (n≥1); —O—(CHMeCH₂O)ₙR (n≥1)); substituted or unsubstituted phosphate group, sulfonyl group, —SO₃H, sulfo group, sulfonic/sulphonic acid, carbamoyl group, primary amine, secondary amine, tertiary amine, carboxamide, cyano group, —NR—O—R, —O—NR, or a salt thereof; and
   wherein R is H, substituted or non-substituted alkyl, and substituted or non-substituted aryl group; and R₁ is —(CH₂)ₙ— (n is 0 to 9); and
b. at least one herbicide selected from the group consisting of atrazine, terbuthylazine, (S)-metolachlor, metolachlor, terbutryn, simazine, dimethenamid, (S)-dimethenamid, flufenacet, acetochlor, alachlor, isoxaflutole, isoxachlortole, mesotrione, sulcotrione, metosulam, flumetsulam, pendimethalin, bromoxynil, bentazone, carfentrazone-ethyl, clomazone, nicosulfuron, rimsulfuron, halosulfuron-methyl, metribuzin, flumiclorac-pentyl, prosulfuron, primisulfuron-methyl, dicamba, fluthiacet-methyl, pyridate, 2,4-D, clopyralide, diflufenzopyr, fluroxypyr, MCPA, MCPB, mecoprop (MCPP), metobenzuron, thifensulfuron-methyl, aclonifen, EPTC, glyphosate, glufosinate, sulfosate, cyanazine, propaquizafop, metamitron, pyramin, phenmedipham, desmedipham, ethofumesate, triasulfuron, chloridazon, lenacil, triallate, fluazifop, sethoxydim, quizalofop, clopyralide, clethodim, oxasulfuron, acifluorfen, benazolin-ethyl, sulfentrazone, chlorimuron-ethyl, cloransulam-methyl, fomesafen, imazamox, imazaquin, imazethapyr, imazapyr, lactofen, fenoxaprop (P-ethyl), thidiazuron, tribufos, trifluralin, dimethachlor, napropamide, quinmerac, metazachlor, carbetamide, dimefuron, propyzamide, ethametsulfuron-methyl, tebutam, fluometuron, prometryn, norflurazon, pyrithiobac-sodium, MSMA, DSMA, diuron, flurochloridone, dithiopyr, thiazopyr, oxyfluorfen, ethalfluralin, clodinafop, amidosulfuron, diclofop-methyl, diflufenican, ethoxysulfuron, fentrazamide, flazasulfuron, florasulam, fluazolate, flucarbazone, flupyrsulfuron-methyl sodium, flurtamone, iodosulfuron, isoproturon, chlortoluron, chlorsulfuron, metsulfuron-methyl, sulfosulfuron, tribenuron-methyl, 2,4-DB, 2,4-DP, bifenox, flamprop-M, imazamethabenz-methyl, ioxynil, tralkoxydim, fluoroglycofen-ethyl, methabenzthiazuron, isoxaben, prosulfocarb, difenzoquat-metilsulfate, pretilachlor, cinosulfuron, fenclorim, bensulfuron-methyl, imazosulfuron, pyrazosulfuron-ethyl, azimsulfuron, esprocarb, mefenacet, molinate, propanil, pyrazolate, cyhalofop-butyl, bispyribac-sodium, pyriminobac-methyl, cafenstrole, oxadiargyl, oxadiazon, bromobutide, MY-100, dymron, NB 061, MK243, HW-52, AC 014, ametryn, hexazinone, asulam, azafenidin, tebuthiuron, ethametsulfuron-methyl, or a combination thereof; and
c. at least one agriculturally acceptable carrier.

According to some embodiments, the invention provides a method of controlling undesired plant growth comprising applying to the locus of said undesired plant growth the agricultural composition according to one or more of the above embodiments.

According to some embodiments, the invention provides a method of controlling undesired plant growth, comprising applying to the locus of the undesired plant growth a herbicidally effective amount of a compound having the structure

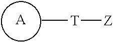

or a salt thereof, wherein:
   A is a cyclopentadiene, benzene and indene scaffold comprising from 1 to 4 heteroatoms; wherein each of the heteroatoms is independently selected from the group consisting of N, S, Se, and O; and wherein one or more of the carbon, nitrogen, sulfur or selenium atoms of the ring are optionally chemically attached to at least one of the groups consisting of: —CX₃, —CX₂R, —COX, —CHO, —COR, —CO₂R, —CONH₂, —CONHR, —CON(R)₂, sulfur ═S, oxygen ═O, —F, —Cl, —Br, and —I, and wherein X is selected from F, Cl, Br and I;
   T is —(CH₂)ₙ—, —OR₁, —SR₁, —SOR₁, —CSR₁, —CS—, —POHR₁, —PO(OR₁)R₁, —CO—, —COR₁, and —COOR₁—;
   Z is H, substituted or non-substituted alkyl, substituted or non-substituted aryl group, —COOH, —COO—, —OH, —O—R, —COOR with saturated or non-saturated alcohol residues with straight, branched, cyclic, aromatic or heteroaromatic chain; —O—(CH₂CH₂O)ₙR (n24 1); —O—(CHMeCH₂O)ₙR (n≥1)); substituted or unsubstituted phosphate group, sulfonyl group, —SO₃H, sulfo group, sulfonic/sulphonic acid, carbamoyl group, primary amine, secondary amine, tertiary amine, carboxamide, cyano group, —NR—O—R, —O—NR, or a salt thereof; and wherein R is H, substituted or non-substituted alkyl, and substituted or non-substituted aryl group; and, R₁ is —(CH₂)ₙ— (n is 0 to 9).

According to some embodiments, the invention provides a method of selectively controlling growth of harmful weeds, comprising applying to a field of a crop an effective amount of the herbicidal composition according to one or more of the above embodiments.

### BRIEF DESCRIPTION OF THE DRAWINGS

FIG. 1 demonstrates pre-emergence herbicidal effect of selected claimed compounds on cereal crops (wheat, maize, rice, barley, sorghum and oat) compared to the effect on Amaranth.

### DETAILED DESCRIPTION OF THE INVENTION

The present invention is now described more fully hereinafter with reference to the accompanying examples, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

According to some embodiments, the invention provides an herbicidal composition comprising a compound or a salt thereof having the structure

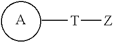

or a salt thereof, wherein:
A is cyclopentadiene, benzene and indene scaffold comprising from 1 to 4 heteroatoms; wherein each of the heteroatoms is independently selected from the group consisting of N, S, Se, and O; and wherein one or more of the carbon, nitrogen, sulfur or selenium atoms of the ring are optionally chemically attached to at least one of the groups consisting of: —SO₂CF₃, —O—SO₂CF₃, —N(R)₃_{⁺}, —SO₂R, —C≡N, —C≡CH, —C≡CR, —CX₃, —CX₂R, —COX, —CHO, —COR, —CO₂R, —CONH₂, —CONHR, —CON(R)₂, —N═O, —N≡N⁺, —N═NR, —CR═NR, —N═C(R)₂, —F, —Cl, —Br, and —I, sulfur ═S, oxygen ═O, and wherein X is selected from F, Cl, Br and I;
T is —(CH₂)ₙ—, —OR₁, —N═CHR₁, —SR₁, —SOR₁, —SOOR₁, —POHR₁, —PO(OR₁)R₁, —C═CR₁, —C═NR₁, —CO—, —COR₁, —CS—, —CSR₁, —COOR₁, and —C=CHR₁;
Z is H, substituted or non-substituted alkyl, substituted or non-substituted aryl group, —COOH, —COO—, —OH, —O—R, —COOR with saturated or non-saturated alcohol residues with straight, branched, cyclic, aromatic or heteroaromatic chain; —O—(CH₂CH₂O)ₙR (n≥1); —O—(CHMeCH₂O)ₙR (n≥1)); substituted or unsubstituted phosphate group, sulfonyl group, carbamoyl group, —SO₃H, sulfo group, sulfonic/sulphonic acid, cyano croup, primary amine, secondary amine, tertiary amine, carboxamide, —NR—O—R, —O—NR₂, hydrazine, —NH—COR, and methanimidamide moiety or a salt thereof; and,
   wherein R is H, substituted or non-substituted alkyl, and substituted or non-substituted aryl group; R₁ is —(CH₂)ₙ— (n is 0 to 9); and, wherein the composition comprises at least one agriculturally acceptable carrier.

According to some embodiments of the above composition, A is an Electron Withdrawing Group (EWG). As used herein, the term “electron withdrawing group” (EWG) refers, without limitation, to an atom or a group that draws electron density from neighboring atoms or aromatic ring, usually by resonance or inductive effects. The non-limiting list of the EWG of the invention includes: trifluoromethanesulfonyl and triflate groups; substituted ammonium groups, such as, without limitation, —NR₃_{⁺} (R is alkyl/s or H); nitro and nitroso groups —NO₂, —N═O; sulfonic acids and sulfonyl groups (—O)—SO₃H, —SO₂R; cyano group, trihalomethyl groups —CX₃(X is F, Cl, Br, I); haloformyl groups —COX (X is F, Cl, Br, I); formyl and acyl groups, —CHO, —COR; (substituted) aminocarbonyl groups —CONH₂, —CONHR, —CONR₂; halo groups —F, —Cl, —Br, —I; Azo groups —N≡N⁺ or —N═NR; Imino group —CR═NR or —N═CR₂.

According to some embodiments of the above composition, T may be, without limitation, —(CH₂)ₙ—, —OR₁, —N═CHR₁, —CS(O)₂R₁, —SR₁, —CS—, —CSR₁, —CO—, —COR₁, —C≡CR₁, —C═NR₁, and —C═CHR₁; wherein R₁ is —(CH₂)ₙ— (n is 0 to 9);

According to some embodiments of the above composition, Z may be, without limitation, H, substituted or non-substituted alkyl, substituted or non-substituted aryl group, carboxyl or salts thereof —COOH or —COO—, hydroxyl —OH, ether —O—R, ester —COOR with saturated or non-saturated alcohol groups with straight, branched, cyclic chain or aromatic/heteroaromatic chain, ethylene- and polyethylene glycol —O—(CH₂CH₂O)ₙR (n≥1, R is H or alkyl), propylene and polypropylene glycol —O—(CHMeCH₂O)ₙR (n≥1, R is H or alkyl), phosphates (substituted and non-substituted), sulfate/sulfonyl, —SO₃H, sulfo group, sulfonic/sulphonic acid, carbamoyl group that consists of primary, secondary, and tertiary amide substituted with straight, branched, cyclic aliphatic chain, cyclic aromatic/heteroaromatic chain, ethylene glycol and polyethylene glycol (as above), propylene glycol, polypropylene glycol (as above), primary amine, secondary amine, tertiary amine, carboxamide, alkoxyamine groups of type —NR—O—R and —O—NR₂, hydrazine, acetamide group —NH—COR, and methanimidamide moiety, or salts thereof.

According to some embodiments of the above composition, z is selected from

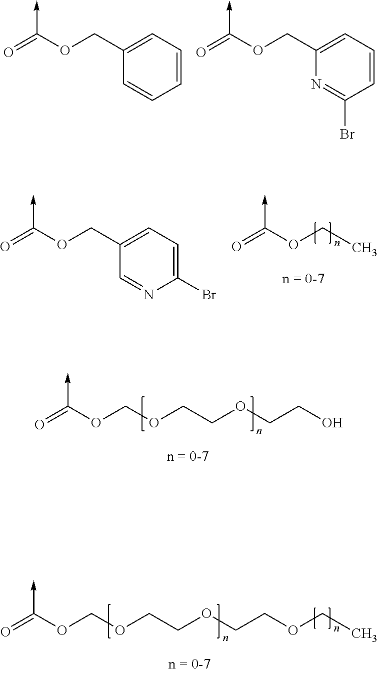

According to some embodiments, the above composition comprises a compound having the structure selected from

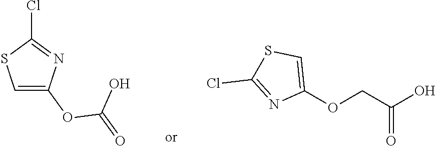 [SMILES: O=C(O)Oc1csc(Cl)n1 ; O=C(O)COc1csc(Cl)n1]

According to some embodiments, the above composition comprises a compound having the structure selected from

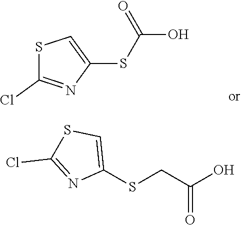

According to some embodiments, the above composition comprises a compound having the structure selected from

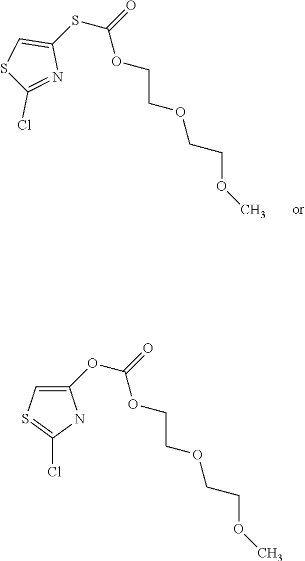

According to some embodiments, the above composition comprises a compound having the structure selected from

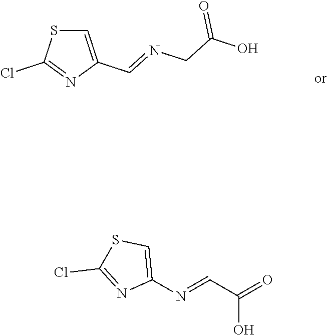 [SMILES: O=C(O)C/N=C/c1csc(Cl)n1 ; O=C(O)/C=N/c1csc(Cl)n1]

According to some embodiments, the above composition comprises a compound having the structure selected from

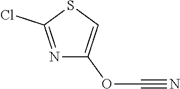

According to some embodiments, the above composition comprises a compound having the structure selected from

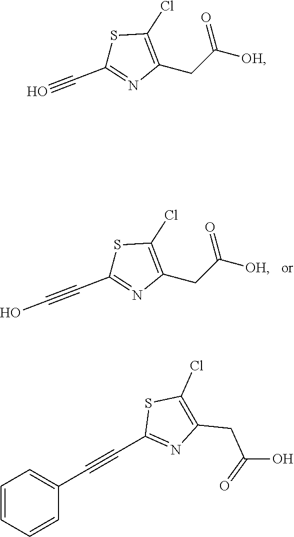

According to some embodiments, the above composition comprises a compound having the structure selected from

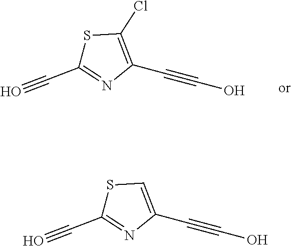

According to some embodiments, the above composition comprises a compound having the structure selected from

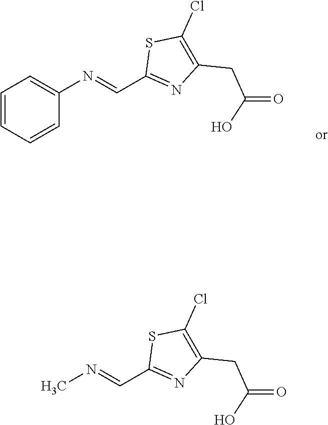 [SMILES: O=C(O)Cc1nc(/C=N/c2ccccc2)sc1Cl ; C/N=C/c1nc(CC(=O)O)c(Cl)s1]

According to some embodiments, the above composition comprises a compound having the structure selected from

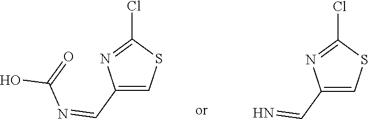 [SMILES: O=C(O)/N=C\c1csc(Cl)n1 ; N=Cc1csc(Cl)n1]

According to some embodiments, the above composition comprises a compound having the structure selected from

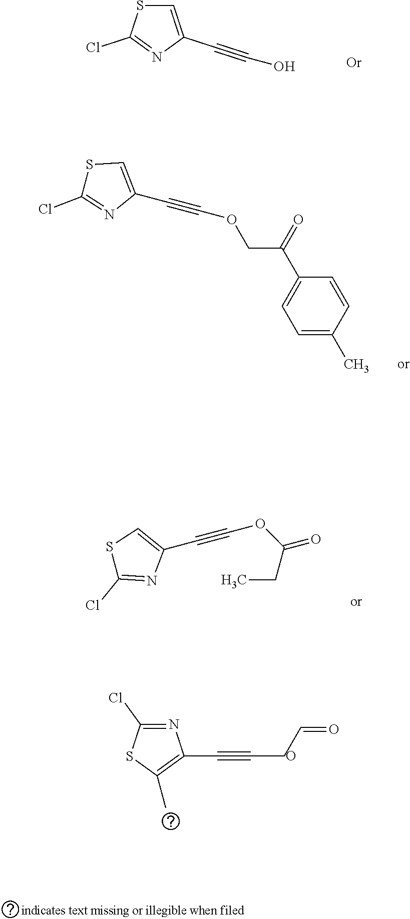

According to some embodiments, the above composition comprises a compound having the structure selected from

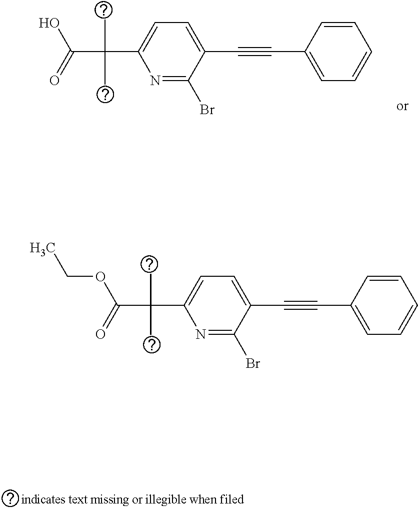 [SMILES: CC(C)(C(=O)O)c1ccc(C#Cc2ccccc2)c(Br)n1 ; CCOC(=O)C(C)(C)c1ccc(C#Cc2ccccc2)c(Br)n1]

According to some embodiments, the above composition comprises a compound having the structure selected from

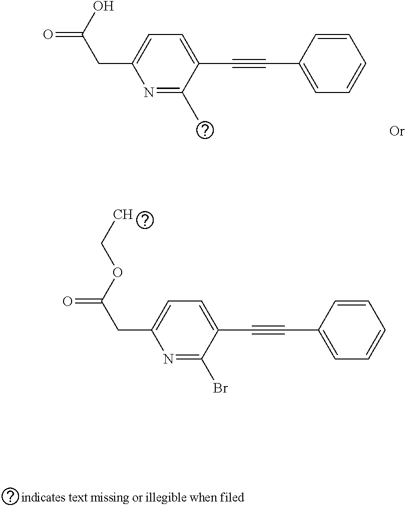 [SMILES: Cc1nc(CC(=O)O)ccc1C#Cc1ccccc1 ; CCOC(=O)Cc1ccc(C#Cc2ccccc2)c(Br)n1]

According to some embodiments, the above composition comprises a compound having the structure selected from

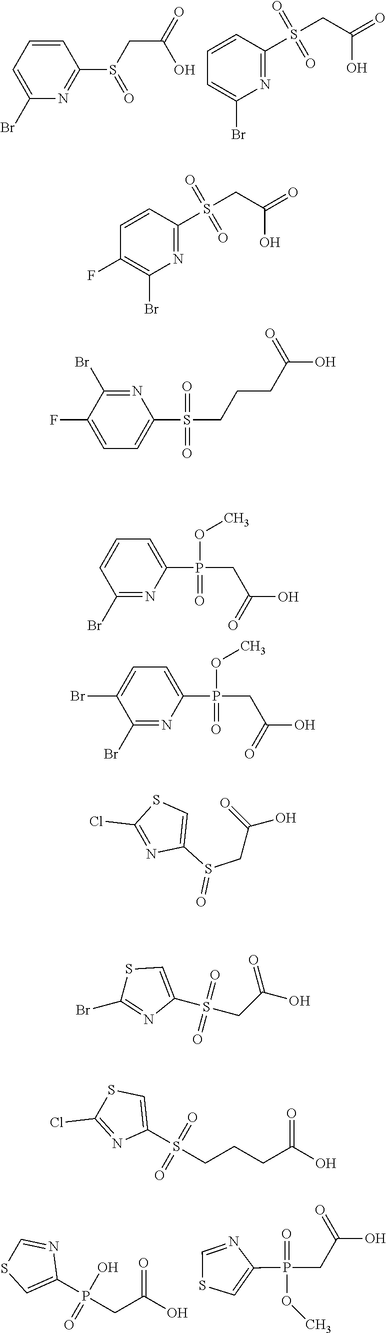

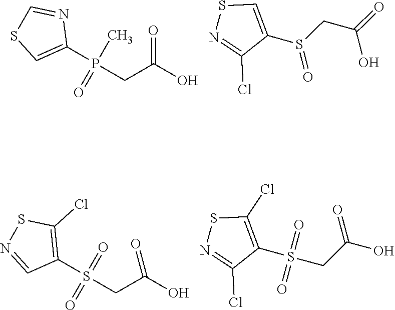

According to some embodiments, the above composition comprises a compound having the structure selected from

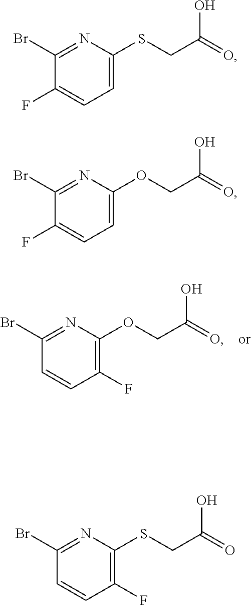 [SMILES: O=C(O)CSc1ccc(F)c(Br)n1 ; O=C(O)COc1ccc(F)c(Br)n1 ; O=C(O)COc1nc(Br)ccc1F ; O=C(O)CSc1nc(Br)ccc1F]

According to some embodiments of the above composition, the herbicidal composition further comprises at least one crop protection agent. In one embodiment, the at least one crop protection agent is selected from the group consisting of fungicide, insecticide, herbicide, and plant growth regulator. In one embodiment, the crop protection agent is herbicide. In another embodiment, the crop protection agent is plant growth regulator. In one embodiment, the at least one crop protection agent is selected from the group consisting of atrazine, terbuthylazine, (S)-metolachlor, metolachlor, terbutryn, simazine, dimethenamid, (S)-dimethenamid, flufenacet, acetochlor, alachlor, isoxaflutole, isoxachlortole, mesotrione, sulcotrione, metosulam, flumetsulam, pendimethalin, bromoxynil, bentazone, carfentrazone-ethyl, clomazone, nicosulfuron, rimsulfuron, halosulfuron-methyl, metribuzin, flumiclorac-pentyl, prosulfuron, primisulfuron-methyl, dicamba, fluthiacet-methyl, pyridate, 2,4-D, clopyralide, diflufenzopyr, fluroxypyr, MCPA, MCPB, mecoprop (MCPP), metobenzuron, thifensulfuron-methyl, aclonifen, EPTC, glyphosate, glufosinate, sulfosate, cyanazine, propaquizafop, metamitron, pyramin, phenmedipham, desmedipham, ethofumesate, triasulfuron, chloridazon, lenacil, triallate, fluazifop, sethoxydim, quizalofop, clopyralide, clethodim, oxasulfuron, acifluorfen, benazolin-ethyl, sulfentrazone, chlorimuron-ethyl, cloransulam-methyl, fomesafen, imazamox, imazaquin, imazethapyr, imazapyr, lactofen, fenoxaprop (P-ethyl), thidiazuron, tribufos, trifluralin, dimethachlor, napropamide, quinmerac, metazachlor, carbetamide, dimefuron, propyzamide, ethametsulfuron-methyl, tebutam, fluometuron, prometryn, norflurazon, pyrithiobac-sodium, MSMA, DSMA, diuron, flurochloridone, dithiopyr, thiazopyr, oxyfluorfen, ethalfluralin, clodinafop, amidosulfuron, diclofop-methyl, diflufenican, ethoxysulfuron, fentrazamide, flazasulfuron, florasulam, fluazolate, flucarbazone, flupyrsulfuron-methyl sodium, flurtamone, iodosulfuron, isoproturon, chlortoluron, chlorsulfuron, metsulfuron-methyl, sulfosulfuron, tribenuron-methyl, 2,4-DB, 2,4-DP, bifenox, flamprop-M, imazamethabenz-methyl, ioxynil, tralkoxydim, fluoroglycofen-ethyl, methabenzthiazuron, isoxaben, prosulfocarb, difenzoquat-metilsulfate, pretilachlor, cinosulfuron, fenclorim, bensulfuron-methyl, imazosulfuron, pyrazosulfuron-ethyl, azimsulfuron, esprocarb, mefenacet, molinate, propanil, pyrazolate, cyhalofop-butyl, bispyribac-sodium, pyriminobac-methyl, cafenstrole, oxadiargyl, oxadiazon, bromobutide, MY-100, dymron, NB 061, MK243, HW-52, AC 014, ametryn, hexazinone, asulam, azafenidin, tebuthiuron, ethametsulfuron-methyl, or a combination thereof.

According to some embodiments, the invention provides a method of controlling undesired plant growth comprising applying to the locus of said undesired plant growth the herbicidal composition according to one or more of the above embodiments.

According to some embodiments, the invention provides a method of controlling undesired plant growth comprising applying to the locus of the undesired plant growth a herbicidally effective amount of a compound having the structure

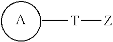

or a salt thereof, wherein:
A is cyclopentadiene, benzene and indene scaffold comprising from 1 to 4 heteroatoms; wherein each of the heteroatoms is independently selected from the group consisting of N, S, Se, and O; and wherein one or more of the carbon, nitrogen, sulfur or selenium atoms of the ring are optionally chemically attached to at least one of the groups consisting of: —SO₂CF₃, —O—SO₂CF₃, —N(R)₃_{⁺}, —SO₂R, —C≡N, —C≡CH, —C≡CR, —CX₃, —CX₂R, —COX, —CHO, —COR, —CO₂R, —CONH₂, —CONHR, —CON(R)₂, —N═O, —N≡N⁺, —N═NR, —CR═NR, —N═C(R)₂, sulfur ═S, oxygen ═O, —F, —Cl, —Br, and —I, and wherein X is selected from F, Cl, Br and I;
T is —(CH₂)ₙ—, —OR₁, —N═CHR₁, —SR₁, —SOR₁, —SOOR₁, —POHR₁, —PO(OR₁)R₁, —C≡CR₁, —C═NR₁, —CO—, —COR₁, —CS—, —CSR₁, —COOR₁, and —C═CHR₁;

Z is H, substituted or non-substituted alkyl, substituted or non-substituted aryl group, —COOH, —COO—, —OH, —O—R, —COOR with saturated or non-saturated alcohol residues with straight, branched, cyclic, aromatic or heteroaromatic chain; —O—(CH₂CH₂O)ₙR (n24 1); —O—(CHMeCH₂O)ₙR (n≥1)); substituted or unsubstituted phosphate group, sulfonyl group, carbamoyl group, —SO₃H, sulfo group, sulfonic/sulphonic acid, cyano croup, primary amine, secondary amine, tertiary amine, carboxamide, —NR—O—R, —O—NR₂, hydrazine, —NH—COR, and methanimidamide moiety or a salt thereof; and,

wherein R is H, substituted or non-substituted alkyl, and substituted or non-substituted aryl group; and, R₁ is —(CH₂)ₙ— (n is 0 to 9).

According to some embodiments of the above method, Z might be selected, without limitation, from the group including:

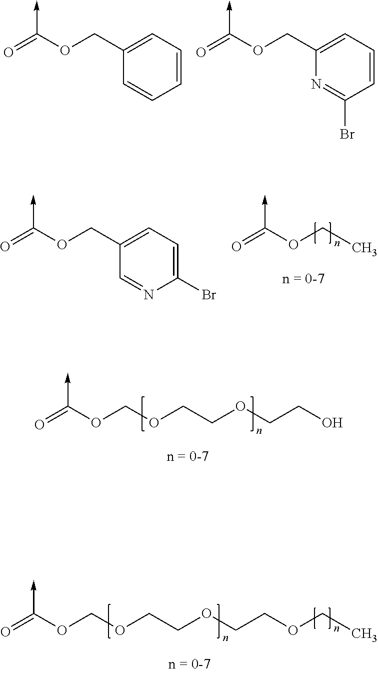

According to some embodiments of the above method, the unlimiting list of compounds, or agriculturally acceptable salts includes:

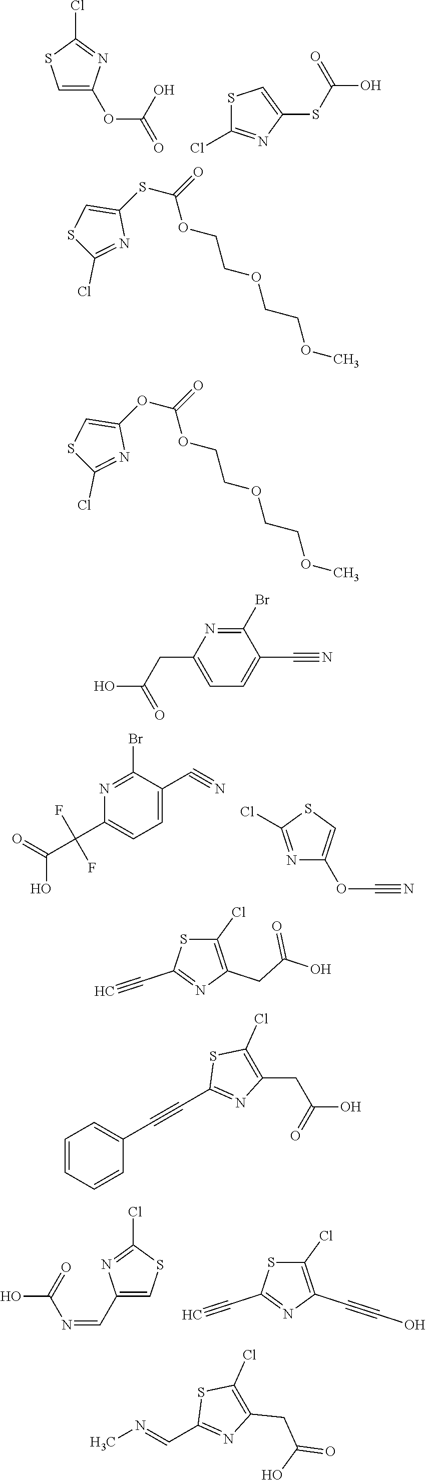

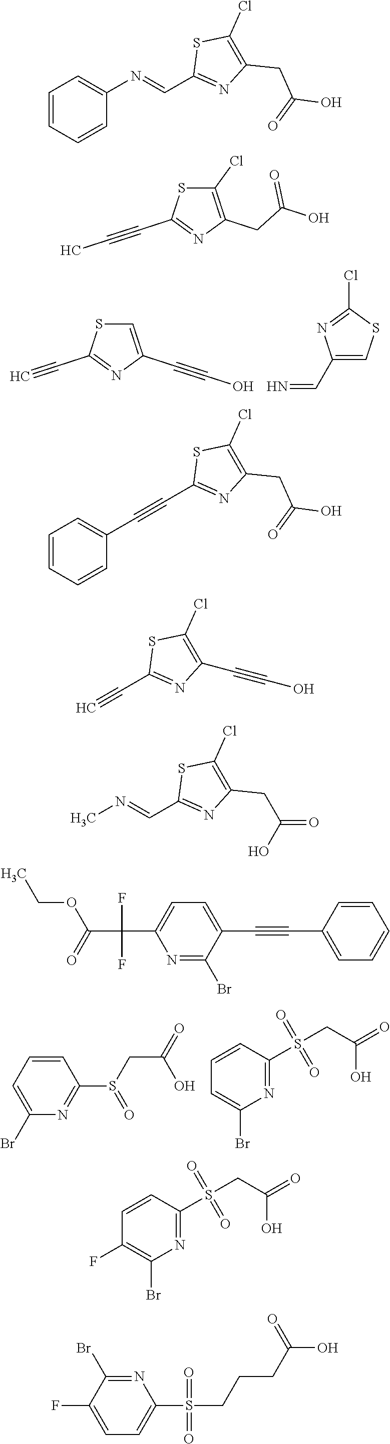

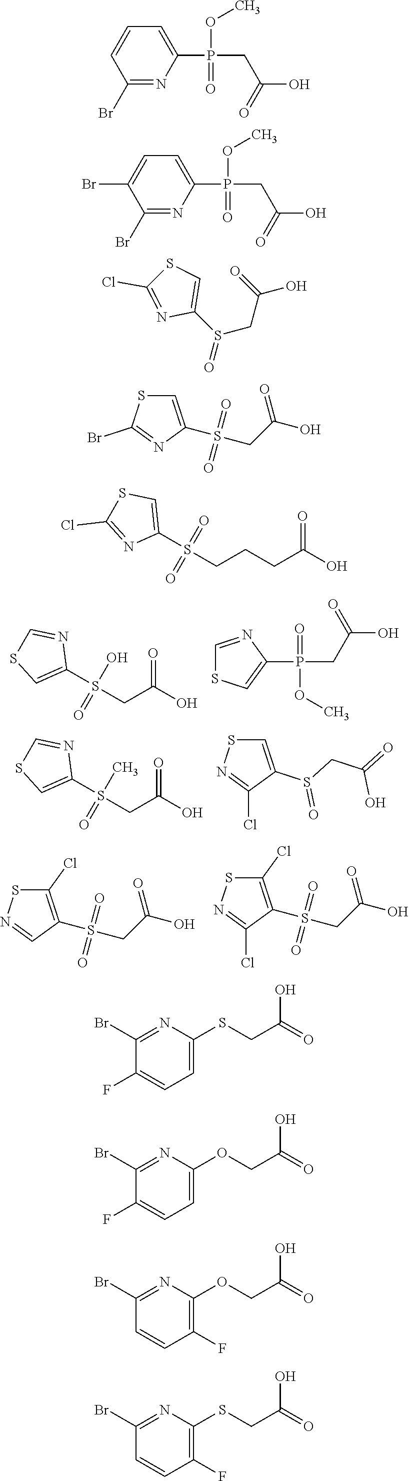

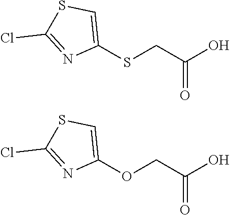 [SMILES: O=C(O)CSc1csc(Cl)n1 ; O=C(O)COc1csc(Cl)n1]

According to some embodiments, the above method further comprises applying to the locus of the undesired plant growth at least one crop protection agent. In one embodiment, the unlimited list of crop protection agents includes, without limitation, herbicide, fungicide, insecticide and plant growth regulator.

According to some embodiments of the above method, the crop protection agent is herbicide.

According to some embodiments of the above method, the crop protection agent is an amino acid synthesis inhibitor herbicide. The non-limiting list of amino acid synthesis inhibitor herbicides includes: sulfonylurea herbicide, imidazolinone herbicide, sulfonamide herbicide, amino acid derivatives, imazamox, imazapic, imazethapyr, imazaquin, imazapyr and imazamethabenz, Chlorimuron, Primisulfuron, Thifensulfuron, Triasulfuron, Nicosulfuron, Metsulfuron, Tribenuron, Rimsulfuron, Triflusulfuron, glyphosate or any combination thereof.

According to some embodiments of the above method, the crop protection agent is a plant growth regulator. The non-limiting list of plant growth regulators includes dicamba, 2,4-D, clopyralid and fluroxypyr.

According to some embodiments, the invention provides a method of controlling undesired plant growth comprising applying to the locus of the undesired plant growth:
a. a first herbicide having the structure

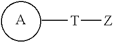

or a salt thereof, wherein:
   A is cyclopentadiene, benzene and indene scaffold comprising from 1 to 4 heteroatoms; wherein each of the heteroatoms is independently selected from the group consisting of N, S, Se, and O; and wherein one or more of the carbon, nitrogen, sulfur or selenium atoms of the ring are optionally chemically attached to at least one of the groups consisting of:—SO₂CF₃, —O—SO₂CF₃, —N(R)₃_{⁺}, —SO₂R, —C≡N, —C≡CH, —C≡CR, —CX₃, —CX₂R, —COX, —CHO, —COR, -CO₂R, —CONH₂, —CONHR, —CON(R)₂, —N═O, —N≡N⁺, —N═NR, —CR═NR, —N═C(R)₂, sulfur ═S, oxygen ═O, —F, —Cl, —Br, and —I, and wherein X is selected from F, Cl, Br and I;
   T is —(CH₂)ₙ—, —OR₁, —N═CHR₁, —SR₁, —SOR₁, —SOOR₁, —POHR₁, —PO(OR₁)R₁, —C≡CR₁, —C═NR₁, —CO—, —COR₁, —CS—, —CSR₁, —COOR₁, or —C═CHR₁;
   Z is H, substituted or non-substituted alkyl, substituted or non-substituted aryl group, —COOH, —COO—, —OH, —O—R, COOR with saturated or non-saturated alcohol residues with straight, branched, cyclic, aromatic or heteroaromatic chain; —O—(CH₂CH₂O)ₙR (n≥1); —O—(CHMeCH₂O)₁R (n≥1)); substituted or unsubstituted phosphate group, sulfonyl group, carbamoyl group, —SO₃H, sulfo group, sulfonic/sulphonic acid, cyano croup, primary amine, secondary amine, tertiary amine, carboxamide, —NR—O—R, —O—NR₂, hydrazine, —NH—COR, and methanimidamide moiety or a salt thereof; and,
   wherein R is H, substituted or non-substituted alkyl, and substituted or non-substituted aryl group; R₁ is —(CH₂)ₙ— (n is 0 to 9); and,
b. a second herbicide, to thereby effectively control the undesired plant growth.

According to some embodiments of the above method, Z may be selected, without limitation, from:

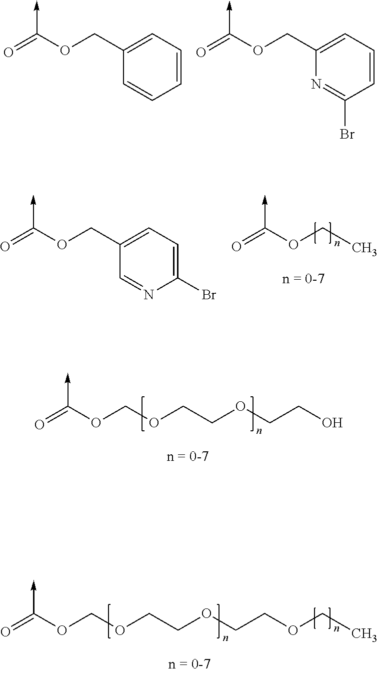

According to some embodiments of the above method, the second herbicide is an amino acid synthesis inhibitor herbicide.

According to some embodiments of the above method, the second herbicide may be selected, without limitation, from sulfonylurea herbicide, imidazolinone herbicide, sulfonamide herbicide, and amino acid derivative.

According to some embodiments of the above method, the non-limiting list of the herbicides of the invention includes: imazamox, imazapic, imazethapyr, imazaquin, imazapyr, imazamethabenz, Chlorimuron, Primisulfuron, Thifensulfuron, Triasulfuron, Nicosulfuron, Metsulfuron, Tribenuron, Rimsulfuron, Triflusulfuron, Glyphosate, atrazine, terbuthylazine, (S)-metolachlor, metolachlor, terbutryn, simazine, dimethenamid, (S)-dimethenamid, flufenacet, acetochlor, alachlor, isoxaflutole, isoxachlortole, mesotrione, sulcotrione, metosulam, flumetsulam, pendimethalin, bromoxynil, bentazone, carfentrazone-ethyl, clomazone, nicosulfuron, rimsulfuron, halosulfuron-methyl, metribuzin, flumiclorac-pentyl, prosulfuron, primisulfuron-methyl, dicamba, fluthiacet-methyl, pyridate, 2,4-D, clopyralide, diflufenzopyr, fluroxypyr, MCPA, MCPB, mecoprop (MCPP), metobenzuron, thifensulfuron-methyl, aclonifen, EPTC, glyphosate, glufosinate, sulfosate, cyanazine, propaquizafop, metamitron, pyramin, phenmedipham, desmedipham, ethofumesate, triasulfuron, chloridazon, lenacil, triallate, fluazifop, sethoxydim, quizalofop, clopyralide, clethodim, oxasulfuron, acifluorfen, benazolin-ethyl, sulfentrazone, chlorimuron-ethyl, cloransulam-methyl, fomesafen, imazamox, imazaquin, imazethapyr, imazapyr, lactofen, fenoxaprop (P-ethyl), thidiazuron, tribufos, trifluralin, dimethachlor, napropamide, quinmerac, metazachlor, carbetamide, dimefuron, propyzamide, ethametsulfuron-methyl, tebutam, fluometuron, prometryn, norflurazon, pyrithiobac-sodium, MSMA, DSMA, diuron, flurochloridone, dithiopyr, thiazopyr, oxyfluorfen, ethalfluralin, clodinafop, amidosulfuron, diclofop-methyl, diflufenican, ethoxysulfuron, fentrazamide, flazasulfuron, florasulam, fluazolate, flucarbazone, flupyrsulfuron-methyl sodium, flurtamone, iodosulfuron, isoproturon, chlortoluron, chlorsulfuron, metsulfuron-methyl, sulfosulfuron, tribenuron-methyl, 2,4-DB, 2,4-DP, bifenox, flamprop-M, imazamethabenz-methyl, ioxynil, tralkoxydim, fluoroglycofen-ethyl, methabenzthiazuron, isoxaben, prosulfocarb, difenzoquat-metilsulfate, pretilachlor, cinosulfuron, fenclorim, bensulfuron-methyl, imazosulfuron, pyrazosulfuron-ethyl, azimsulfuron, esprocarb, mefenacet, molinate, propanil, pyrazolate, cyhalofop-butyl, bispyribac-sodium, pyriminobac-methyl, cafenstrole, oxadiargyl, oxadiazon, bromobutide, MY-100, dymron, NB 061, MK243, HW-52, AC 014, ametryn, hexazinone, asulam, azafenidin, tebuthiuron, and ethametsulfuron-methyl.

According to some embodiments of the above method, the method of controlling undesired plant growth further comprises applying a third herbicide or a plant growth regulator.

According to some embodiments, the invention provides a composition for controlling undesired plant growth comprising a mixture of:
a. a compound or a salt thereof having the structure

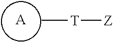

or a salt thereof, wherein:
   A is cyclopentadiene, benzene and indene scaffold comprising from 1 to 4 heteroatoms; wherein each of the heteroatoms is independently selected from the group consisting of N, S, Se, and O; and wherein one or more of the carbon, nitrogen, sulfur or selenium atoms of the ring are optionally chemically attached to at least one of the groups consisting of: —SO₂CF₃, —O—SO₂CF₃, —N(R)₃_{⁺}, —SO₂R, —C≡N, —C≡CH, —C≡CR, —CX₃, —CX₂R, —COX, —CHO, —COR, —CO₂R, —CONH₂, —CONHR, —CON(R)₂, —N═O, —N≡N⁺, —N═NR, —CR═NR, —N═C(R)₂, sulfur ═S, oxygen ═O, —F, —Cl, —Br, and —I, and wherein X is selected from F, Cl, Br and I;
   T is —(CH₂)ₙ—, —OR₁, —N═CHR₁, —SR₁, —SOR₁, —SOOR₁, —S(O)₂R₁, —POHR₁, —PO(OR₁)R₁, —C≡CR₁, —C═NR₁, —CO—, —COR₁, —CS—, —CSR₁, —COOR₁, or —C═CHR₁;
   Z is H, substituted or non-substituted alkyl, substituted or non-substituted aryl group, —COOH, —COO—, —OH, —O—R, —COOR with saturated or non-saturated alcohol residues with straight, branched, cyclic, aromatic or heteroaromatic chain; —O—(CH₂CH₂O)ₙR (n≥1); —O—(CHMeCH₂O)ₙR (n≥1)); substituted or unsubstituted phosphate group, sulfonyl group, carbamoyl group, —SO₃H, sulfo group, sulfonic/sulphonic acid, cyano croup, primary amine, secondary amine, tertiary amine, carboxamide, —NR—O—R, —O—NR₂, hydrazine, —NH—COR, and methanimidamide moiety or a salt thereof; and,
   wherein R is H, substituted or non-substituted alkyl, and substituted or non-substituted aryl group; R₁ is —(CH₂)ₙ— (n is 0 to 9); and,
b. at least one herbicide selected from the group consisting of atrazine, terbuthylazine, (S)-metolachlor, metolachlor, terbutryn, simazine, dimethenamid, (S)-dimethenamid, flufenacet, acetochlor, alachlor, isoxaflutole, isoxachlortole, mesotrione, sulcotrione, metosulam, flumetsulam, pendimethalin, bromoxynil, bentazone, carfentrazone-ethyl, clomazone, nicosulfuron, rimsulfuron, halosulfuron-methyl, metribuzin, flumiclorac-pentyl, prosulfuron, primisulfuron-methyl, dicamba, fluthiacet-methyl, pyridate, 2,4-D, clopyralide, diflufenzopyr, fluroxypyr, MCPA, MCPB, mecoprop (MCPP), metobenzuron, thifensulfuron-methyl, aclonifen, EPTC, glyphosate, glufosinate, sulfosate, cyanazine, propaquizafop, metamitron, pyramin, phenmedipham, desmedipham, ethofumesate, triasulfuron, chloridazon, lenacil, triallate, fluazifop, sethoxydim, quizalofop, clopyralide, clethodim, oxasulfuron, acifluorfen, benazolin-ethyl, sulfentrazone, chlorimuron-ethyl, cloransulam-methyl, fomesafen, imazamox, imazaquin, imazethapyr, imazapyr, lactofen, fenoxaprop (P-ethyl), thidiazuron, tribufos, trifluralin, dimethachlor, napropamide, quinmerac, metazachlor, carbetamide, dimefuron, propyzamide, ethametsulfuron-methyl, tebutam, fluometuron, prometryn, norflurazon, pyrithiobac-sodium, MSMA, DSMA, diuron, flurochloridone, dithiopyr, thiazopyr, oxyfluorfen, ethalfluralin, clodinafop, amidosulfuron, diclofop-methyl, diflufenican, ethoxysulfuron, fentrazamide, flazasulfuron, florasulam, fluazolate, flucarbazone, flupyrsulfuron-methyl sodium, flurtamone, iodosulfuron, isoproturon, chlortoluron, chlorsulfuron, metsulfuron-methyl, sulfosulfuron, tribenuron-methyl, 2,4-DB, 2,4-DP, bifenox, flamprop-M, imazamethabenz-methyl, ioxynil, tralkoxydim, fluoroglycofen-ethyl, methabenzthiazuron, isoxaben, prosulfocarb, difenzoquat-metilsulfate, pretilachlor, cinosulfuron, fenclorim, bensulfuron-methyl, imazosulfuron, pyrazosulfuron-ethyl, azimsulfuron, esprocarb, mefenacet, molinate, propanil, pyrazolate, cyhalofop-butyl, bispyribac-sodium, pyriminobac-methyl, cafenstrole, oxadiargyl, oxadiazon, bromobutide, MY-100, dymron, NB 061, MK243, HW-52, AC 014, ametryn, hexazinone, asulam, azafenidin, tebuthiuron, ethametsulfuron-methyl, or a combination thereof; and
c. at least one agriculturally acceptable carrier.

According to some embodiments, the invention provides a composition for controlling undesired plant growth comprising a mixture of:
a. a compound or a salt thereof having the structure

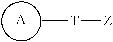

or a salt thereof, wherein:
   A is cyclopentadiene, benzene and indene scaffold comprising from 1 to 4 heteroatoms; wherein each of the heteroatoms is independently selected from the group consisting of N, S, Se, and O; and wherein one or more of the carbon, nitrogen, sulfur or selenium atoms of the ring are optionally chemically attached to at least one of the groups consisting of: —SO₂CF₃, —O—SO₂CF₃, —N(R)₃_{⁺}, —SO₂R, —C≡N, —C≡CH, —C≡CR, —CX₃, —CX₂R, —COX, —CHO, —COR, —CO₂R, —CONH₂, —CONHR, —CON(R)₂, —N═O, —N≡N⁺, —N═NR, —CR═NR, —N═C(R)₂, sulfur ═S, oxygen ═O, —F, —Cl, —Br, and —I, and wherein X is selected from F, Cl, Br and I;
   T is —(CH₂)ₙ—, —OR₁, —N═CHR₁, —SR₁, —SOR₁, —SOOR₁, —S(O)₂R₁, —POHR₁, —PO(OR₁)R₁, —C≡CR₁, —C═NR₁, —CO—, —COR₁, —CS—, —CSR₁, —COOR₁, and —C═CHR₁;
   Z is H, substituted or non-substituted alkyl, substituted or non-substituted aryl group, —COOH, —COO—, —OH, —O—R, COOR with saturated or non-saturated alcohol residues with straight, branched, cyclic, aromatic or heteroaromatic chain; —O—(CH₂CH₂O)ₙR (n≥1); —O—(CHMeCH₂O)ₙR (n≥1)); substituted or unsubstituted phosphate group, sulfonyl group, carbamoyl group, —SO₃H, sulfo group, sulfonic/sulphonic acid, cyano croup, primary amine, secondary amine, tertiary amine, carboxamide, —NR—O—R, —O—NR₂, hydrazine, —NH—COR, and methanimidamide moiety or a salt thereof; and,
   wherein R is H, substituted or non-substituted alkyl, and substituted or non-substituted aryl group; R₁ is —(CH₂)ₙ—(n is 0 to 9);
b. at least one plant growth regulator; and
c. at least one agriculturally acceptable carrier.

According to some embodiments, the invention provides a compound having the structure

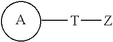

or a salt thereof, wherein:
   A is cyclopentadiene, benzene and indene scaffold comprising from 1 to 4 heteroatoms; wherein each of the heteroatoms is independently selected from the group consisting of N, S, Se, and O; and wherein one or more of the carbon, nitrogen, sulfur or selenium atoms of the ring are optionally chemically attached to at least one of the groups consisting of:—SO₂CF₃, —O—SO₂CF₃, —N(R)₃_{⁺}, —SO₂R, —C≡N, —C≡CH, —C≡CR, —CX₃, —CX₂R, —COX, —CHO, —COR, —CO₂R, —CONH₂, —CONHR, —CON(R)₂, —N=O, —N≡N⁺, —N═NR, —CR═NR, —N═C(R)₂, sulfur ═S, oxygen ═O, —F, —Cl, —Br, and —I, and wherein X is selected from F, Cl, Br and I;
   T is —(CH₂)ₙ—, —OR₁, —N═CHR₁, —SR₁, —SOR₁, —SOOR₁, —POHR₁, —PO(OR₁)R₁, —C≡CR₁, —C═NR₁, —CO—, —COR₁, —CS—, —CSR₁, —COOR₁, and —C═CHR₁;
   Z is H, substituted or non-substituted alkyl, substituted or non-substituted aryl group, —COOH, —COO—, —OH, —O—R, —COOR with saturated or non-saturated alcohol residues with straight, branched, cyclic, aromatic or heteroaromatic chain; —O—(CH₂CH₂O)ₙR (n≥1); —O—(CHMeCH₂O)ₙR (n≥1)); substituted or unsubstituted phosphate group, sulfonyl group, carbamoyl group, —SO₃H, sulfo group, sulfonic/sulphonic acid, cyano croup, primary amine, secondary amine, tertiary amine, carboxamide, —NR—O—R, —O—NR₂, hydrazine, —NH—COR, and methanimidamide moiety or a salt thereof; and, wherein R is H, substituted or non-substituted alkyl, and substituted or non-substituted aryl group; and R₁ is —(CH₂)ₙ—(n is 0 to 9).

According to some embodiments, method of controlling amino acid content in a plant or a plant part thereof comprises applying an effective amount of the herbicidal composition according to one or more of the above embodiments.

According to some embodiments, the non-limiting list of the above compounds or salts thereof includes:

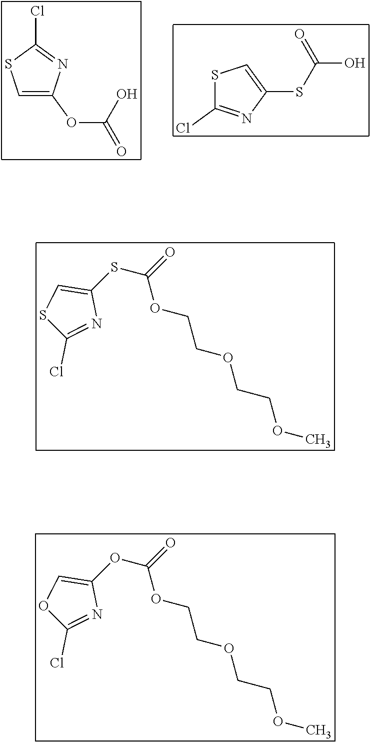 [SMILES: O=C(O)Oc1csc(Cl)n1 ; O=C(O)Sc1csc(Cl)n1 ; COCCOCCOC(=O)Sc1csc(Cl)n1 ; COCCOCCOC(=O)Oc1coc(Cl)n1]

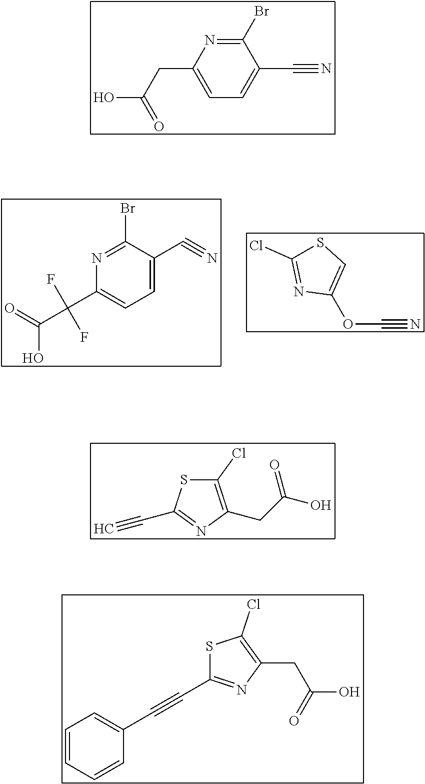 [SMILES: N#Cc1ccc(CC(=O)O)nc1Br ; N#Cc1ccc(C(F)(F)C(=O)O)nc1Br ; N#COc1csc(Cl)n1 ; C#Cc1nc(CC(=O)O)c(Cl)s1 ; O=C(O)Cc1nc(C#Cc2ccccc2)sc1Cl]

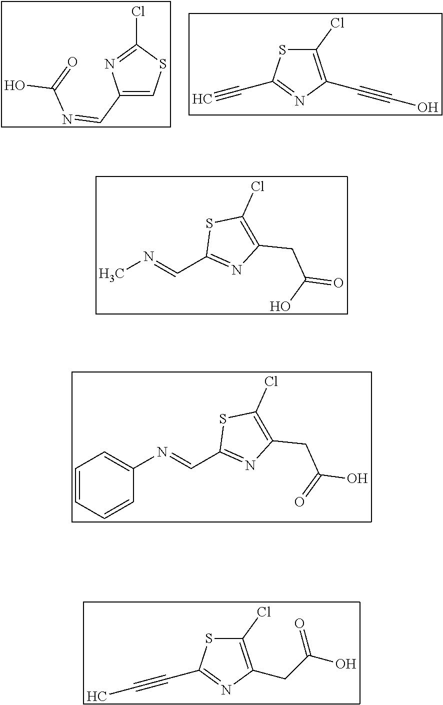 [SMILES: O=C(O)/N=C\c1csc(Cl)n1 ; C#Cc1nc(C#CO)c(Cl)s1 ; C/N=C/c1nc(CC(=O)O)c(Cl)s1 ; O=C(O)Cc1nc(/C=N/c2ccccc2)sc1Cl ; CC#Cc1nc(CC(=O)O)c(Cl)s1]

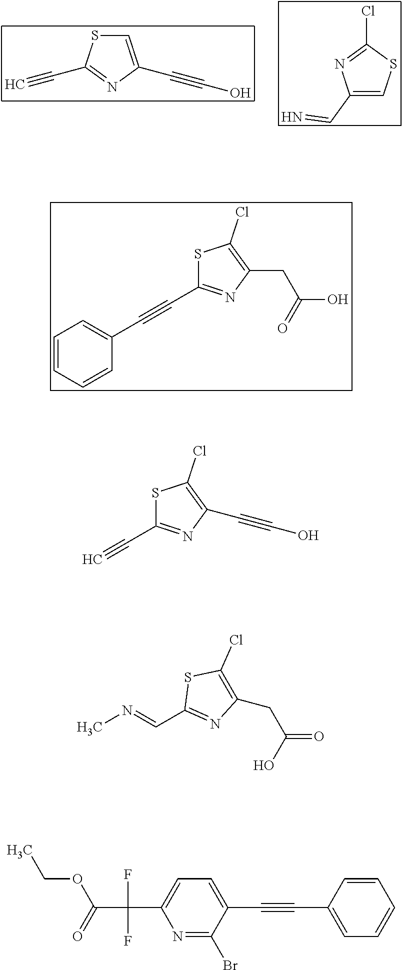

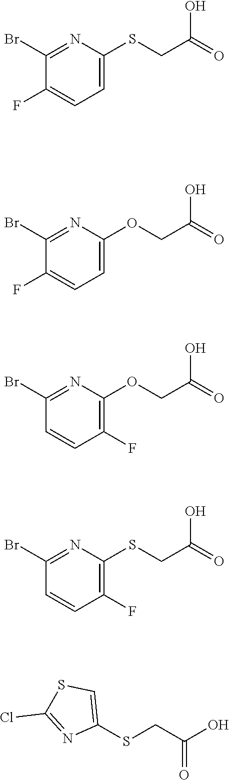 [SMILES: O=C(O)CSc1ccc(F)c(Br)n1 ; O=C(O)COc1ccc(F)c(Br)n1 ; O=C(O)COc1nc(Br)ccc1F ; O=C(O)CSc1nc(Br)ccc1F ; O=C(O)CSc1csc(Cl)n1]

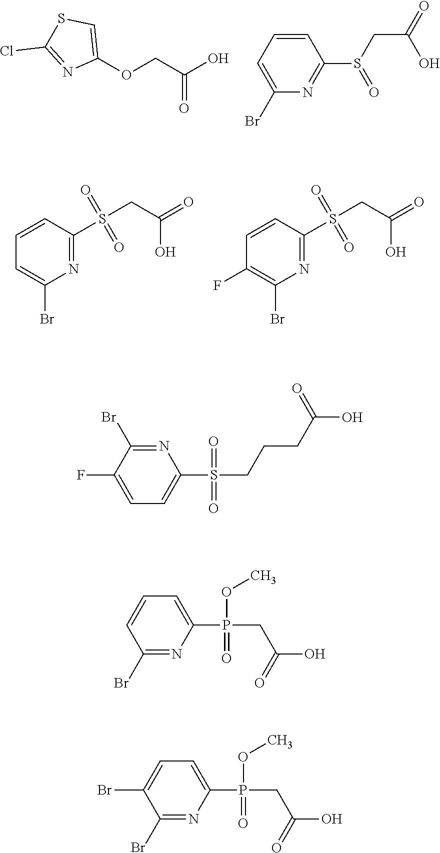

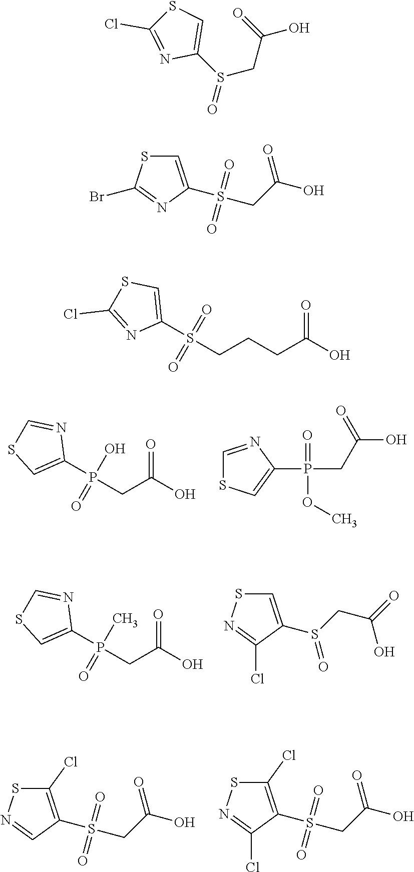

According to some embodiments, the invention provides a method of controlling plant growth comprising applying to the plant or a plant part thereof, an effective amount of the herbicidal composition or the compound according to one or more of the above embodiments.

According to some embodiments, the invention provides a method of controlling growth of plants at least partially resistant to herbicides selected from the HPAC 9 group (Inhibitors of EPSP synthesis), 2 (Acetolactate Synthase (ALS) inhibitors), and 4 (Plant growth regulators or synthetic auxins), comprising applying to the plant or a plant part thereof, an effective amount of the herbicidal composition or the compound according to one or more of the above embodiments.

According to some embodiments of the above method, the locus of the undesired vegetation is a field of a crop.

According to some embodiments of the above method, the crop is selected from the group consisting of maize, wheat, rice, barley, sorghum, and oats.

According to some embodiments of the above method, the herbicidal composition is applied pre-emergence.

According to some embodiments of the above method, the herbicidal composition is applied post-emergence.

According to some embodiments, the invention provides a method of selectively controlling growth of harmful and/or undesirable weeds, comprising applying to a field of a crop an effective amount of the herbicidal composition or compound, according to one or more of the above embodiments. As used herein, the term “selectively controlling” refers, without limitation, to a situation when growth of the harmful and/or undesirable weeds is affected by the compositions according to the embodiments of the invention, without affecting growth of desirable vegetation, such as, without limitation, crops. According to some embodiments, the weeds are considered as relevant to maize, wheat, rice, barley, sorghum, and/or oat cultivation.

According to some embodiments of the above method the herbicidal composition is applied pre-emergence.

According to some embodiments of the above method, the herbicidal composition is applied post-emergence.

According to some embodiments of the above method, wherein the crop is selected from the group consisting of maize, wheat, rice, barley, sorghum, and oats.

According to some embodiments of the above methods, the effective amount of the of the herbicidal composition or compound, according to one or more of the above embodiments is in a range of between 50 gr/Ha to 2000 gr/Ha, when applied pre-emergence.

According to some embodiments of the above methods, the effective amount of the of the herbicidal composition or compound, according to one or more of the above embodiments is 50 gr/Ha, 100 gr/Ha, 150 gr/Ha, 200 gr/Ha, 250 gr/Ha, 300 gr/Ha, 350 gr/Ha, 400 gr/Ha, 450 gr/Ha, 500 gr/Ha, 550 gr/Ha, 600 gr/Ha, 650 gr/Ha, 700 gr/Ha, 750 gr/Ha, 800 gr/Ha, 850 gr/Ha, 900 gr/Ha, 950 gr/Ha, 1000 gr/Ha, 1050 gr/Ha, 1100 gr/Ha, 1150 gr/Ha, 1200 gr/Ha, 1250 gr/Ha, 1300 gr/Ha, 1350 gr/Ha, 1400 gr/Ha, 1450 gr/Ha, 1500 gr/Ha, 1550 gr/Ha, 1600 gr/Ha, 1650 gr/Ha, 1700 gr/Ha, 1750 gr/Ha, 1800 gr/Ha, 1850 gr/Ha, 1900 gr/Ha, 1950 gr/Ha, 2000 gr/Ha, when applied pre-emergence.

According to some embodiments of the above methods, the effective amount of the of the herbicidal composition or compound, according to one or more of the above embodiments is in a range of between 125 gr/Ha to 2000 gr/Ha, when applied post-emergence.

According to some embodiments of the above methods, the effective amount of the of the herbicidal composition or compound, according to one or more of the above embodiments is, 125 gr/Ha, 150 gr/Ha, 175 gr/Ha, 200 gr/Ha, 225 gr/Ha, 250gr/Ha, 275 gr/Ha, 300 gr/Ha, 325 gr/Ha, 350 gr/Ha, 375 gr/Ha, 400 gr/Ha, 425 gr/Ha, 450 gr/Ha, 475 gr/Ha, 500 gr/Ha, 525 gr/Ha, 550 gr/Ha, 575 gr/Ha, 600 gr/Ha, 625 gr/Ha, 650 gr/Ha, 675 gr/Ha, 700 gr/Ha, 725 gr/Ha, 750 gr/Ha, 775 gr/Ha, 800 gr/Ha, 825 gr/Ha, 850 gr/Ha, 875 gr/Ha, 900 gr/Ha, 925 gr/Ha, 950 gr/Ha, 975 gr/Ha, 1000 gr/Ha, 1025 gr/Ha, 1050 gr/Ha, 1075 gr/Ha, 1100 gr/Ha, 1125 gr/Ha, 1150 gr/Ha, 1175 gr/Ha, 1200 gr/Ha, 1225 gr/Ha, 1250 gr/Ha, 1275 gr/Ha, 1300 gr/Ha, 1325 gr/Ha, 1350 gr/Ha, 1375 gr/Ha, 1400 gr/Ha, 1425 gr/Ha, 1450 gr/Ha, 1475 gr/Ha, 1500 gr/Ha, 1525 gr/Ha, 1550 gr/Ha, 1575 gr/Ha, 1600 gr/Ha, 1625 gr/Ha, 1650 gr/Ha, 1675 gr/Ha, 1700 gr/Ha, 1725 gr/Ha, 1750 gr/Ha, 1775 gr/Ha, 1800 gr/Ha, 1825 gr/Ha, 1850 gr/Ha, 1875 gr/Ha, 1900 gr/Ha, 1925 gr/Ha, 1950 gr/Ha, 1975 gr/Ha, 2000 gr/Ha, when applied pre-emergence.

### Results

As appears from the experimental data, NCAADs of the invention have strong herbicidal activity. This is as opposed to initial assumption that these compounds can only act as inert structural analogues of herbicidal non-coded AAs (NCAAs). Moreover, surprisingly, the NCAADs of the invention demonstrate 10 to 100 times higher herbicidal activity then corresponding NCAAs.

Numerous NCAADs showing herbicidal activity both in foliar and soil applications suggesting effective absorption by leaves, shoots, and roots. While the corresponding NCAAs, only exhibited herbicidal activity in soil application.

### Example 1: Derivatives of Non-Coded Amino Acids With Herbicidal Activity

All compounds were synthetized de novo with purity ≥95%. Structure demonstrated with LCMS and H-NMR. Table 1 comprises a non-exclusive list of derivatives of non-coded heterocyclic amino acids exemplifying claims.

TABLE 1

| ID | Chemical name |
|---|---|
| 5647 | 3-[(2,5-dibromothiophene-3-sulfonyl)amino]propanoic |
| | acid |
| 5648 | N-(2-aminoethyl)-2,5-dibromothiophene-3-sulfonamide |
| | hydrochloride |
| 5839 | (2,4-dichlorothiophen-3-yl)acetic acid |
| 5847 | (2,5-dichlorothiophene-3-sulfonyl)acetic acid |
| 5850 | ethyl 2-[(2-chloro-1,3-thiazol-4-yl)sulfanyl]acetate |
| 5851 | (2-chloro-1,3-thiazol-4-sulfonyl)acetic acid |
| 6687 | 2-(6-bromopyridine-2-sulfinyl)acetic acid |
| 6688 | [(6-bromopyridin-2-yl)sulfanyl]acetic acid |
| 6689 | (6-bromopyridine-2-sulfinyl)acetic acid |
| 6693 | ethyl [(6-bromopyridin-2-yl)sulfanyl]acetate |
| 6694 | butyl [(3-bromo-6-chloropyridin-2- |
| | yl)sulfanyl]acetate |
| 6695 | butyl (3-bromo-6-chloropyridine-2-sulfinyl)acetate |
| 6700 | butyl [(5,6-dibromopyridin-2-yl)sulfanyl]acetate |
| 6701 | N1-(6-bromopyridin-2-yl)butane-1,4-diamine |
| 6702 | 2-[(6-bromopyridin-2-yl)amino]acetic acid |
| 6703 | butyl (5,6-dibromopyridine-2-sulfinyl)acetate |
| 6709 | N1-(5,6-dibromopyridin-2-yl)butane-1,4-diamine |
| 7106 | octyl [(6-bromopyridin-2-yl)sulfanyl]acetate |
| 7109 | 2-[(4-chloro-1,3-thiazol-2-yl)sulfanyl]acetic acid |
| 7110 | ethyl 2-[(4-chloro-1,3-thiazol-2-yl)amino]acetate |
| 7111 | 4-chloro-N-[3-(4-chlorophenyl)propyl]-1,3-thiazol-2- |
| | amine |
| 7112 | ethyl [(4-chloro-1,3-thiazol-2-yl)sulfanyl]acetate |
| 7113 | N1-(6-bromopyridin-2-yl)propane-1,3-diamine |
| 7114 | ethyl 2-[(6-bromo-5-fluoropyridin-2- |
| | yl)sulfanyl]acetate |
| 7117 | ethyl (6-bromo-5-fluoropyridin-2-yl)acetate |
| 7118 | ethyl [(6-bromo-5-fluoropyridin-2-yl)oxy]acetate |
| 7118 | ethyl 2-[(6-bromo-5-fluoropyridin-2-yl)oxy]acetate |
| 7119 | ethyl 2-[(2, 5-dichloro-1,3-thiazol-4- |
| | yl)sulfanyl]acetate |
| 7121 | N-{3-[(6-bromopyridin-2-yl)amino]propyl}-2- |
| | phenylacetamide |
| 7122 | 2-[(5,6-dibromopyridin-2-yl)amino]acetic acid |
| 7123 | N-{4-[(6-bromopyridin-2-y1)amino]butyl}-2- |
| | phenylacetamide |
| 7124 | [(3,6-dibromopyridin-2-yl)sulfanyl]acetic acid |
| 7125 | 2-[(4-chloro-1,3-thiazol-2-yl)amino]acetic acid |
| 7130 | N-{4-[(5,6-dibromopyridin-2-yl)amino]butyl} acetamide |
| 7131 | ethyl 4-(cyanomethyl)-1H-pyrrole-3-carboxylate |
| 7133 | tert-butyl {4-[(4-chloro-1,3-thiazol-2- |
| | yl)amino]butyl}carbamate |
| 7134 | tert-butyl {2-[(4-chloro-1,3-thiazol-2- |
| | yl)amino]ethyl}carbamate |
| 7137 | (2,5-dichloro-1,3-thiazole-4-sulfinyl)acetic acid |
| 7138 | (2,5-dichloro-1,3-thiazol-4-sulfonyl)acetic acid |
| 7139 | 2-(2-ethoxyethoxy)ethyl [(5,6-dibromopyridin-2- |
| | yl)sulfanyl]acetate |
| 7145 | 4-(2,5-dichloro-1,3-thiazol-4-yl)but-3-ynoic acid |
| 7147 | 2-[(2,4-dichlorothiophen-3-yl)amino]acetic acid |
| | hydrochloride |
| 7148 | 2-[(6-bromo-5-fluoropyridin-2-yl)amino]acetic acid |
| 7151 | [(6-bromo-5-fluoropyridin-2-yl)sulfanyl]acetic acid |
| 7153 | ethyl 2-[(2,4-dichlorothiophen-3-yl)amino]acetate |
| 7156 | 2-(2-ethoxyethoxy)ethyl 2-[(6-bromo-5-fluoropyridin- |
| | 2-yl)oxy]acetate |
| 7157 | [(6-bromo-5-fluoropyridin-2-yl)oxy]acetic acid |
| 7158 | [5-chloro-3-(methanesulfinyl)thiophen-2-yl]acetic |
| | acid |
| 7160 | ethyl 2-[(6-bromo-3-fluoropyridin-2- |
| | yl)sulfanyl]acetate |
| 7163 | 2-[(2-chloropyridin-3-yl)oxy]acetic acid |
| 7164 | 2-[(3,6-dibromopyridin-2-yl)amino]acetic acid |
| 7165 | 5-[(6-bromo-5-fluoropyridin-2-yl)amino]pentanoic |
| | acid |
| 7167 | methyl 2-(2-chloro-5-cyano-1,3-thiazol-4-yl)acetate |
| 7168 | methyl 2-(5-cyano-1,3-thiazol-4-yl)acetate |
| 7169 | 2-[(5,6-dibromo-3-fluoropyridin-2-yl)amino]acetic |
| | acid |
| 7170 | 5-[(5,6-dibromo-3-fluoropyridin-2-yl)amino]pentanoic |
| | acid |
| 7172 | 2-(2-ethoxyethoxy)ethyl [(5,6-dibromopyridin-2- |
| | yl)oxy]acetate |
| 7174 | 4-[(5,6-dibromopyridin-2-yl)sulfanyl]butanoic acid |
| 7178 | 3-[(5,6-dibromopyridin-2-yl)sulfanyl]propanoic acid |
| 7182 | 2-[(5,6-dibromopyridin-2-yl)sulfanyl]propanoic acid |
| 7186 | (2,5-dichlorothiophen-3-yl)methanesulfonic acid |
| 7187 | 5-[(5,6-dibromopyridin-2-yl)sulfanyl]pentanoic acid |
| 7188 | ethyl dichloro(2-chloro-1,3-thiazol-4-yl)acetate |
| 7189 | S-(2-chloro-1,3-thiazol-4-yl) O-ethyl carbonothioate |
| 7194 | 2-chloro-1,3-thiazole-4-carboxylic acid |
| 7195 | 2-[N-(5-chloro-1,3-thiazol-4-yl)acetamido]acetic |
| | acid |

### Example 2: Effect of Derivatives of Non-Coded Amino Acids on Germination and Early Development of Plants/Weeds/Polycots

Materials: Seeds of Lettuce R. (Super-Jericho, non-sterilized) were obtained from Ben Shahar Moshe Ltd., 99% purity. The test compounds listed in Table 3 below were synthesized de novo or purchased from different vendors. Bioassays: Filter Paper Bioassays with Lettuce seeds were performed following Bertin et al. 2009 with modifications as described below. Seeds were placed on Whatman no. 1 filter paper (Whatman, Middlesex, U.K.) in Petri dishes (10 seeds per plate) with 2.0 ml of an aqueous solution of test material in a concentration ranging from 0 (control) to 1 mmol/l. The Petri dishes were arranged in a tray tilted at a 45-degree angle and were kept in the dark for 48 hours. Then trays with plates were transferred to the growth chamber with a 6/18 dark/light cycle for four days. Each experiment was performed in at least two repeats.

The development of plants' radicles and shoots was visually assessed six days after the beginning of the test to determine the Minimal Effective Concentration (MEC) of a test material. In the context of the invention, MEC is defined as the lowest concentration level of a test material that caused deviation (malformation of radicle or shoots). MEC is expressed in an arbitrary unit as an activity score, as presented in Table 2:

| MEC | Activity score |
|---|---|
| Higher than 0.1 mM | 0 |

| | | |
|---|---|---|
| 0.1 | mM | 3 |
| 0.01 | mM | 4 |
| 0.001 | mM | 5 |
| <0.001 | mM | 6 |

TABLE 3
represents herbicidal activity of selected derivatives of Non-Coded
Amino Acids on germination and early development of the plants

| ID | MEC, score |
|---|---|
| 5850 | 4 |
| 5851 | 5 |
| 6688 | 5 |
| 6693 | 5 |
| 6694 | 5 |
| 6700 | 5 |
| 6702 | 5 |
| 6703 | 4 |
| 6705 | 4 |
| 6706 | 5 |
| 6709 | 4 |
| 7106 | 4 |
| 7109 | 4 |
| 7112 | 4 |
| 7114 | 6 |
| 7117 | 4 |
| 7118 | 6 |
| 7118 | 5 |
| 7119 | 4 |
| 7121 | 4 |
| 7122 | 5 |
| 7123 | 4 |
| 7124 | 5 |
| 7125 | 4 |
| 7131 | 4 |
| 7137 | 4 |
| 7138 | 4 |
| 7139 | 5 |
| 7145 | 5 |
| 7147 | 4 |
| 7148 | 4 |
| 7151 | 6 |
| 7156 | 5.5 |
| 7157 | 5 |
| 7160 | 6 |
| 7165 | 4 |
| 7167 | 4 |
| 7168 | 4 |
| 7169 | 5 |
| 7170 | 5 |
| 7172 | 5 |
| 7174 | 6 |
| 7178 | 5 |
| 7182 | 4 |
| 7187 | 5 |
| 7188 | 3 |
| 7189 | 4 |
| 7194 | 3 |

The results provided in Example 2 (table 3) exemplified the effects of the claimed compounds on a plant's germination and early development. The effect of the tested compounds in the germination test usually correlates well with the compounds' herbicidal activity in both pre-and post-emergence applications.

### Example 3: Herbicidal activity of Selected Derivatives of Non-Coded Amino Acids on the Development of Weeds

Weed seeds were sawn in 0.3-liter pots containing medium-heavy clay-loam soil from organic fields (55% clay, 23% silt, 20% sand, 2% organic matter, pH 7.1). The experiment was conducted in a completely randomized design. Each treatment was replicated three times (3 pots). Each compound was typically tested on a weed panel which included Amaranth, Abutilon, Malva, Silybum, and Sinapis.

The herbicides were applied at doses from 0 (control) to 5 mg/pot. The herbicides were applied on grown plants at stage 2-3 true leaves (post-emergence treatment). The plants were grown in the mesh house during the entire experiment. Plant development at 6, 12, 19, 26, and 32 days after application (DAA) was evaluated on a scale of 0 to 5, in which 0 represents dead plants, and 5 represents healthy plants similar or not different from Control. In the context of the invention, the herbicidal effect was expressed as a score calculated as an average of assessments of plants' vigor, height, and leaf color. The results of the experiments are presented in Table 4 below.

TABLE 4
Average Herbicidal Effect of Derivatives
of Non-Coded Amino Acids on potted weeds:

| Weed/ID | 7114 | 7118 | 7156 | 7157 |
|---|---|---|---|---|
| Amaranth | 3 | 1 | 1 | 0 |
| Abutilon | 5 | 4 | 1 | 3 |
| Malva | 4 | 1 | 1 | 1 |
| Silybum | 5 | 1 | 1 | 1 |
| Sinapis | 4 | 1 | 1 | 1 |
| Trifolium | 4 | 2 | 2 | 2 |
| Lactuca | 5 | 3 | 2 | 2 |
| Lettuce | | 5 | 0 | 1 |
| Solanum | | 0 | 0 | 0 |
| Verbesina | | 2 | 1 | 0 |

### Example 5: Selectivity of Select Derivatives of Non-Coded Amino Acids to Cereals

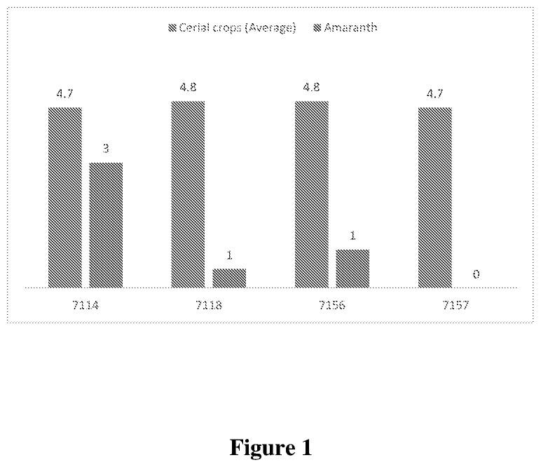

Selectivity to cereals of claimed compounds was exemplified using the compounds: 7114, 6693, 5839, 6688. The tests were performed on cereal crops such as wheat, maize, rice, barley, sorghum, and oats (FIG. 1). The test plants were grown and treated as described in Example 4 (post-emergence foliar applications). The herbicidal effect was assessed using the Damage Score as detailed in Example 4.

Selectivity of tested compounds to cereals was observed up to the doses 5 kg a.i./Ha, 0.05% and 0.5 kg a.i./Ha in foliar, drench, and soil applications, respectively.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof. As used herein the terms “comprises”, “comprising”, “includes”, “including”, “having” and their conjugates mean “including but not limited to”. The term “consisting of” means “including and limited to”.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Whenever the term “about” is used, it is meant to refer to a measurable value such as an amount, a temporal duration, and the like, and is meant to encompass variations of ±20%, ±10%, ±5%, ±1%, or ±0.1% from the specified value, as such variations are appropriate to perform the disclosed methods.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

As used herein the terms “comprises”, “comprising”, “includes”, “including”, “having” and their conjugates mean “including but not limited to”.

The term “consisting of” means “including and limited to”.

As used herein, the singular form “a”, “an” and “the” include plural references unless the context clearly dictates otherwise. For example, the term “a compound” or “at least one compound” may include a plurality of compounds, including mixtures thereof.

It will be understood that when an element is referred to as being “on,” “attached” to, “connected” to, “coupled” with, “contacting,” etc., another element, it can be directly on, attached to, connected to, coupled with and/or contacting the other element or intervening elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

As will be appreciated by those of skill in the art, the compounds of the various formulas disclosed herein may contain chiral centers, e.g., asymmetric carbon atoms. Thus, the present disclosure is concerned with the synthesis of both: (i) racemic mixtures of the active compounds, and (ii) enantiomeric forms of the active compounds. The resolution of racemates into enantiomeric forms and racemization of optically active enantiomeric form can be done in accordance with known procedures in the art. Geometric isomers of double bonds and the like may also be present in the compounds disclosed herein, and all such stable isomers are included within the present disclosure unless otherwise specified. Also included in the compounds of the disclosure are tautomers (e.g., tautomers of triazole and/or imidazole) and rotamers. All chains defined by the formulas herein which include three or more carbons may be saturated or unsaturated unless otherwise indicated.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

An “optionally substituted” group refers to a functional group in which one or more bonds to a hydrogen atom contained therein are replaced by a bond to non-hydrogen or non-carbon atoms, provided that normal valences are maintained and that the substitution results in a stable compound. Substituted groups also include groups in which one or more bonds to a carbon(s) or hydrogen(s) atom are replaced by one or more bonds, including double or triple bonds, to a heteroatom. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different. In choosing the compounds of the present invention, one of ordinary skill in the art will recognize that the various substituents are to be chosen in conformity with well-known principles of chemical structure connectivity.

As used herein, “H” refers to a hydrogen atom. “C” refers to a carbon atom. “N” refers to a nitrogen atom. “O” refers to an oxygen atom. “Halo” refers to F, Cl, Br or I. The term “hydroxy,” as used herein, refers to an —OH moiety. “Br” refers to a bromine atom. “Cl” refers to a chlorine atom. “I” refers to an iodine atom. “F” refers to a fluorine atom. An “acyl group” is intended to mean a group —C(O)—R, where R is a suitable substituent, for example, an acetyl group, a propionyl group, a butyroyl group, a benzoyl group, or an alkylbenzoyl group. “Alkyl,” as used herein, refers to a straight or branched chain hydrocarbon containing from 1 or 2 to 10 or 20 or more carbon atoms (e.g., C2, C3, C4, C5, C6, C7, C8, C9, C10, C11, C12, C13, C14, C15, etc.). In some embodiments the alkyl can be a lower alkyl. “Lower alkyl” refers to straight or branched chain alkyl having from 1 to 3, or from 1 to 5, or from 1 to 8 carbon atoms. Representative examples of alkyl include, but are not limited to, methyl, ethyl, n-propyl, iso-propyl, n-butyl, sec-butyl, iso-butyl, tert- butyl, n-pentyl, isopentyl, neopentyl, n-hexyl, 3-methylhexyl, 2, 2-dimethylpentyl, 2, 3-dimethylpentyl, n-heptyl, n-octyl, n-nonyl, n-decyl, and the like. As used herein, the identification of a carbon number range, e.g., C1-C12 alkyl, is intended to include each of the component carbon number moieties within such range, so that each intervening carbon number and any other stated or intervening carbon number value in that stated range is encompassed, such that sub-ranges of carbon number within specified carbon number ranges may independently be specified. For example, C1-C12 alkyl is intended to include methyl, ethyl, propyl, butyl, pentyl, hexyl, heptyl, octyl, nonyl, decyl, undecyl and dodecyl, including straight chain as well as branched groups, as noted above, and the carbon number range C1-C12 alkyl may also be more restrictively specified as sub-ranges such as C1-C4 alkyl, C2-C8 alkyl, C2-C4 alkyl, C3-C5 alkyl, or any other sub-range within the broader carbon number range. In addition, ranges of carbon numbers specifically excluding a carbon number or numbers are contemplated, as are sub-ranges excluding either or both of carbon number limits of specified ranges. As generally understood by those of ordinary skill in the art, “saturation” refers to the state in which all available valence bonds of an atom (e.g., carbon) are attached to other atoms. Similarly, “unsaturation” refers to the state in which not all the available valence bonds are attached to other atoms; in such compounds the extra bonds usually take the form of double or triple bonds (usually with carbon). For example, a carbon chain is “saturated” when there are no double or triple bonds present along the chain or directly connected to the chain (e.g., a carbonyl), and is “unsaturated” when at least one double or triple bond is present along the chain or directly connected to the chain (e.g., a carbonyl). Further, the presence or absence of a substituent depending upon chain saturation will be understood by those of ordinary skill in the art to depend upon the valence requirement of the atom or atoms to which the substituent binds (e.g., carbon). “Alkenyl,” as used herein, refers to a straight or branched chain hydrocarbon containing from 1 or 2 to 10 or 20 or more carbons, and containing at least one carbon-carbon double bond, formed structurally, for example, by the replacement of two hydrogens. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like.

“Alkynyl,” as used herein, refers to a straight or branched chain hydrocarbon group containing from 1 or 2 to 10 or 20 or more carbon atoms, and containing at least one carbon-carbon triple bond. Representative examples of alkynyl include, but are not limited, to acetylenyl, 1-propynyl, 2-propynyl, 3-butynyl, 2-pentynyl, 1-butynyl and the like. The term “cycloalkyl,” as used herein, refers to a saturated cyclic hydrocarbon group containing from 3 to 8 carbons or more.

As understood in the art, the term “optionally substituted” indicates that the specified group is either unsubstituted or substituted by one or more suitable substituents. A “substituent” that is “substituted” is an atom or group which takes the place of a hydrogen atom on the parent chain or cycle of an organic molecule. “Heterocycle,” as used herein, refers to a monocyclic, bicyclic or tricyclic ring system. Monocyclic heterocycle ring systems are exemplified by any 5 to 9-membered ring containing 1, 2, 3, or 4 heteroatoms independently selected from the group consisting of: O, N, and S. “Aryl” as used herein refers to a ring system having one or more aromatic rings.

Representative examples of aryl include azulenyl, indanyl, indenyl, naphthyl, phenyl, tetrahydronaphthyl, and the like. “Heteroaryl” means a cyclic, aromatic hydrocarbon in which one or more carbon atoms have been replaced with heteroatoms (e.g., N, O or S). If the heteroaryl group contains more than one heteroatom, the heteroatoms may be the same or different. “Alkoxy,” as used herein, refers to an alkyl group, as defined herein, appended to the parent molecular moiety through an oxy group, as defined herein. An “amine” or “amino” is intended to mean the group —NH₂. “Primary amines” have one of three hydrogen atoms replaced by an alkyl or aromatic group. “Secondary amines” have two organic substituents bound to the nitrogen together with one hydrogen. “Tertiary amines” have three organic substituents bound to the nitrogen. An “amide” as used herein, refers to a functional group having a carbonyl group (C═O) linked to a nitrogen atom (N), or an organic compound that contains this group, generally depicted as:

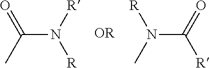

wherein, R and R′ can independently be any covalently linked atom or atoms. The term “oxo,” as used herein, refers to a ═O moiety. The term “oxy,” as used herein, refers to a —O—moiety. “Nitro” refers to the organic compound functional group —NO₂. “Carbonyl” is a functional group having a carbon atom double-bonded to an oxygen atom (—C═0). “Carboxy” as used herein refers to a —COOH functional group, also written as —CO₂H or —(C═O)—OH.

It will be understood that the compounds, compositions and methods provided herein may be further specified in some embodiments by provisos or limitations excluding specific substituents, groups, moieties, structures, ingredients, steps, or conditions, as applicable, in relation to various broader specifications and exemplifications set forth herein. “Agriculturally acceptable carriers” of the invention include, without limitation, adjuvants, mixers, enhancers, etc. beneficial for application of the chemical formula. Suitable carriers should not be phytotoxic to valuable crops, particularly at the concentrations employed in applying the compositions for selective weed control in the presence of crops and should not react chemically with the compounds of the chemical formula herein or other composition ingredients. Such mixtures can be designed for application directly to weeds or their locus or can be concentrates or formulations which are normally diluted with additional carriers and adjuvants before application. They may include inert or active components and can be solids, such as, for example, dusts, granules, water dispersible granules, or wettable powders, or liquids, such as, for example, emulsifiable concentrates, solutions, emulsions or suspensions. Suitable agricultural carriers useful in preparing herbicidal compositions of the present invention are well known to those skilled in the art. For example, liquid carriers that can be employed include water, toluene, xylene, petroleum naphtha, crop oil, acetone, methyl ethyl ketone, cyclohexanone, trichloroethylene, perchloroethylene, ethyl acetate, amyl acetate, butyl acetate, propylene glycol monomethyl ether and diethylene glycol monomethyl ether, methanol, ethanol, isopropanol, amyl alcohol, ethylene glycol, propylene glycol, glycerine, and the like. Water is generally the carrier of choice for the dilution of concentrates. Suitable solid carriers include talc, pyrophyllite clay, silica, attapulgus clay, kieselguhr, chalk, diatomaceous earth, lime, calcium carbonate, bentonire clay, Fuller's earth, cotton seed hulls, wheat flour, soybean flour, pumice, wood flour, walnut shell flour, lignin, and the like.

It is frequently desirable to incorporate one or more surface-active agents into the compositions of the present invention. Such surface-active agents are advantageously employed in both solid and liquid compositions, especially those designed to be diluted with carrier before application. The surface-active agents can be anionic, cationic or nonionic in character and can be employed as emulsifying agents, wetting agents, suspending agents, or for other purposes. Typical surface active agents include salts of alkyl sulfates, such as diethanolammonium lauryl sulfate; alkylarylsulfonate salts, such as calcium dodecylbenzenesulfonate; alkylphenol-alkylene oxide addition products, such as nonylphenol-C.sub.18 ethoxylate; alcohol-alkylene oxide addition products, such as tridecyl alcohol-C.sub.16 ethorylate; soaps, such as sodium stearate; alkylnaphthalenesulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in herbicidal compositions include antifoam agents, compatibilizing agents, sequestering agents, neutralizing agents and buffers, corrosion inhibitors, dyes, odorants, penetrations aids, spreading agents, sticking agents, dispersing agents, thickening agents, freeze point depressants, antimicrobial agents, and the like. The compositions can also contain other compatible components, for example, other herbicides, plant growth regulants, fungicides, insecticides, and the like and can be formulated with liquid fertilizers or solid, particulate fertilizer carriers such as ammonium nitrate, urea and the like.

“Agriculturally acceptable salt” is intended to mean a salt that retains the biological effectiveness of the free acids and bases of a specified compound and that is not biologically or otherwise undesirable. Examples of agriculturally acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

Throughout this application, various embodiments of this invention may be presented in a range format. It should be understood that the description in range format is merely for convenience and brevity and should not be construed as an inflexible limitation on the scope the invention. of Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, agricultural, biological, and biochemical arts.

As used herein the term “plant growth regulator” refers but not limited to a compound, either natural or synthetic, that modifies or controls one or 1 more specific physiological processes within a plant.

As used herein the term “plant” refers but not limited to whole plants, ancestors and progeny of the plants and plant parts, including seeds, shoots, stems, roots (including tubers), and plant cells, tissues and organs. The plant may be in any form including suspension cultures, embryos, meristematic regions, callus tissue, leaves, gametophytes, sporophytes, pollen, and microspores.

As used herein the term “crop protection agent” refers but not limited to an agent which is a pesticide (or a mixture of more than one pesticide) or a plant growth regulator.

As used herein the term “pesticide” refers to, but not limited to a chemical or biological agent that deters, incapacitates, kills, or otherwise discourages pests.

Compounds of this invention will generally be used as a herbicidal active ingredient in a composition. As used herein the term “composition” refers but not limited to a formulation, with at least one additional component selected from the oup consisting of surfactants, solid diluents and liquid diluents, which serves as a carrier. The formulation or composition ingredients are selected to be consistent with the physical properties of the active ingredient, mode of application and environmental factors such as oil type, moisture and temperature. Useful compositions may include both liquid and solid formulation. Liquid formulations may include solutions (including emulsifiable concentrates), suspensions, emulsions (including microemulsions, oil-in-water emulsions, flowable concentrates and/or suspoemulsions) and alike, which optionally can be thickened into gels. The general types of aqueous liquid compositions are soluble concentrate, suspension concentrate, capsule suspension, concentrated emulsion, microemulsion, oil-in-water emulsion, flowable concentrate and suspo-emulsion. The general types of nonaqueous liquid compositions are emulsifiable concentrate, microemulsifiable concentrate, dispersible concentrate and oil dispersion.

The general types of solid formulations are dusts, powders, granules, pellets, prills, astilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively, the entire formulation of active ingredient can be encapsulated (or “overcoated”). Encapsulation can control or delay release of the active ingredient. Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil.

Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting.

The formulations will typically contain effective amounts of active ingredient, diluent and surfactant within the following approximate ranges which add up to 100 percent by eight.

The compounds of the invention have (both preemergent and postemergent) herbicidal activity. As used herein the term “controlling undesired plant growth” refers to killing or injuring the vegetation or reducing its growth.

The compounds and compositions of the invention can be usefully applied by a variety of methods involving contacting a herbicidally effective amount of a compound of the invention, or a composition comprising said compound and at least one of a surfactant, a solid diluent or a liquid diluent, to the foliage or other part of the undesired plant or to the environment of the undesired plant growth such as the soil or water in which the undesired plant is growing or which surrounds the seed or other propagule of the undesired plant.

A herbicidally effective amount of the compounds of this invention is determined by a number of factors: formulation selected, method of application, amount and type of undesired plant growth present, growing conditions, etc. In general, a herbicidally effective amount of compounds of this invention is about 0.001 to 20 kg/ha with a preferred range of about 0.004to 1 kg/ha. One skilled in the art can easily determine the herbicidally effective amount necessary for the desired level of weed control.

The compounds of the invention are applied, typically in a form of formulated composition, to a locus comprising desired vegetation (e.g., crops) and undesired plant growth (i.e. weeds), both of which may be seeds, seedlings and/or larger plants, in contact with a growth medium (e.g., soil). In this locus, a composition comprising the compounds of the invention can be directly applied to a plant or any part of a plant thereof, particularly of the undesired plant growth, and/or to the growth medium in contact with the plant.

Compounds of this invention can also be mixed with one or more other biologically active compounds or agents including herbicides, herbicide safeners, fungicides, insecticides, nematocides, bactericides, acaricides, plant growth regulators inhibitors and rooting stimulants, such as insect molting chemosterilants, semiochemicals, repellents, attractants, pheromones, feeding stimulants, plant nutrients, other biologically active compounds or bacteria, virus or fungi to form a multi-component pesticide giving broader spectrum of agricultural protection. Mixtures of the compounds of the invention with other herbicides can broaden the spectrum of activity against additional weed species and suppress the proliferation of any resistant biotypes.

Throughout this application various publications, published patent applications and published patents are referenced. The disclosures of these publications in their entireties are hereby incorporated by reference into this application in order to more fully describe the state of the art to which this invention pertains.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

1. An herbicidal composition comprising a compound having the structure 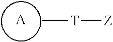
or a salt thereof, wherein:
A is a cyclopentadiene, or benzene scaffold comprising from 1 to 4 heteroatoms; wherein each of the heteroatoms is independently selected from the group consisting of N, S, and; and wherein one or more of the carbon, nitrogen, sulfur or selenium atoms of the ring are optionally chemically attached to at least one of —CX₃, —CX₂R, —COX, —CHO, —COR, —CO₂R, —CONH₂, —CONHR, ═S, ═O, —F, —Cl, —Br, and —I, and wherein X is selected from F, Cl, Br and I; and
T is —(CH₂)ₙ—, —OR₁—, —SR₁—, —SOR₁—, —CSR₁—, —CS—, —POHR₁—, —PO(OR₁)R₁—, —CO—, or —C(O)R₁—;
Z is H, substituted or non-substituted alkyl, substituted or non-substituted aryl group, —COOH, —COO—, —OH, —O—R, —COOR with saturated or non-saturated alcohol residues with, straight, branched, cyclic, aromatic or heteroaromatic chain, —O—(CH₂CH₂O)ₙR (n≥1), —O—(CHMeCH₂O)ₙR (n≥1)), substituted or unsubstituted phosphate group, cyano group, carbamoyl group, primary amine, secondary amine, tertiary amine, carboxamide, —NR—O—R, —O—NR, or a salt thereof; and,
wherein R is H, substituted or non-substituted alkyl, or substituted or non-substituted aryl group; and, wherein R₁ is —(CH₂)ₙ—(n is 0 to 9); and, wherein the composition comprises at least one agriculturally acceptable carrier.

2. The herbicidal composition of claim 1, wherein Z is selected from

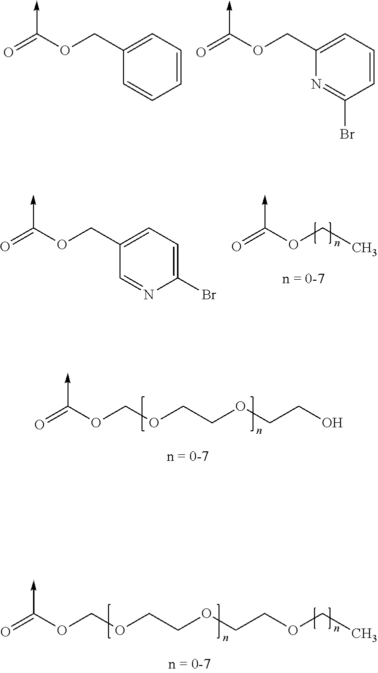

3. The herbicidal composition of claim 1, comprising the compound having the structure

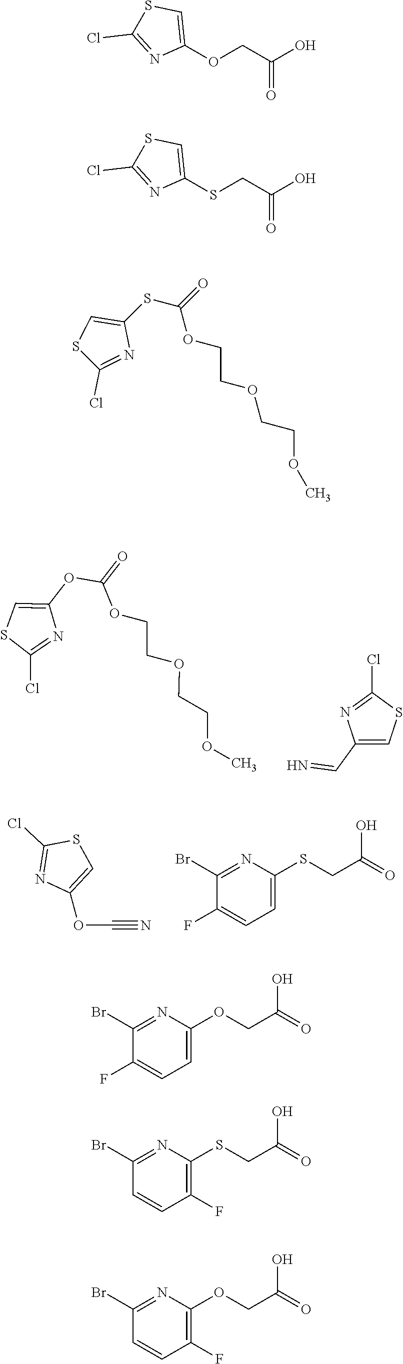

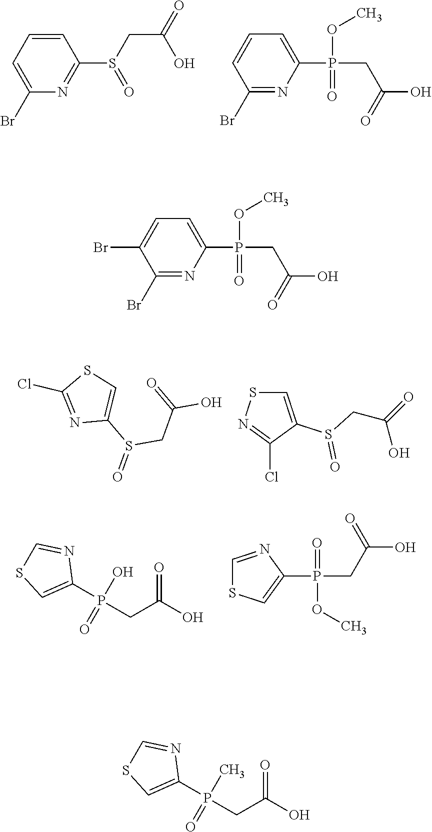

4. The herbicidal composition of claim 1, further comprising at least one crop protection agent.

5. The herbicidal composition of claim 4, wherein the at least one crop protection agent is selected from the group consisting of fungicide, insecticide, herbicide, and plant growth regulator.

6. The herbicidal composition of claim 5, wherein the at least one crop protection agent is selected from the group consisting of atrazine, terbuthylazine, (S)-metolachlor, metolachlor, terbutryn, simazine, dimethenamid, (S)-dimethenamid, flufenacet, acetochlor, alachlor, isoxaflutole, isoxachlortole, mesotrione, sulcotrione, metosulam, flumetsulam, pendimethalin, bromoxynil, bentazone, carfentrazone-ethyl, clomazone, nicosulfuron, rimsulfuron, halosulfuron-methyl, metribuzin, flumiclorac-pentyl, prosulfuron, primisulfuron-methyl, dicamba, fluthiacet-methyl, pyridate, 2,4-D, clopyralide, diflufenzopyr, fluroxypyr, MCPA, MCPB, mecoprop (MCPP), metobenzuron, thifensulfuron-methyl, aclonifen, EPTC, glyphosate, glufosinate, sulfosate, cyanazine, propaquizafop, metamitron, pyramin, phenmedipham, desmedipham, ethofumesate, triasulfuron, chloridazon, lenacil, triallate, fluazifop, sethoxydim, quizalofop, clopyralide, clethodim, oxasulfuron, acifluorfen, benazolin-ethyl, sulfentrazone, chlorimuron-ethyl, cloransulam-methyl, fomesafen, imazamox, imazaquin, imazethapyr, imazapyr, lactofen, fenoxaprop (P-ethyl), thidiazuron, tribufos, trifluralin, dimethachlor, napropamide, quinmerac, metazachlor, carbetamide, dimefuron, propyzamide, ethametsulfuron-methyl, tebutam, fluometuron, prometryn, norflurazon, pyrithiobac-sodium, MSMA, DSMA, diuron, flurochloridone, dithiopyr, thiazopyr, oxyfluorfen, ethalfluralin, clodinafop, amidosulfuron, diclofop-methyl, diflufenican, ethoxysulfuron, fentrazamide, flazasulfuron, florasulam, fluazolate, flucarbazone, flupyrsulfuron-methyl sodium, flurtamone, iodosulfuron, isoproturon, chlortoluron, chlorsulfuron, metsulfuron-methyl, sulfosulfuron, tribenuron-methyl, 2,4-DB, 2,4-DP, bifenox, flamprop-M, imazamethabenz-methyl, ioxynil, tralkoxydim, fluoroglycofen-ethyl, methabenzthiazuron, isoxaben, prosulfocarb, difenzoquat-metilsulfate, pretilachlor, cinosulfuron, fenclorim, bensulfuron-methyl, imazosulfuron, pyrazosulfuron-ethyl, azimsulfuron, esprocarb, mefenacet, molinate, propanil, pyrazolate, cyhalofop-butyl, bispyribac-sodium, pyriminobac-methyl, cafenstrole, oxadiargyl, oxadiazon, bromobutide, MY-100, dymron, NB 061, MK243, HW-52, AC 014, ametryn, hexazinone, asulam, azafenidin, tebuthiuron, ethametsulfuron-methyl, or a combination thereof.

7. A compound having the structure selected from 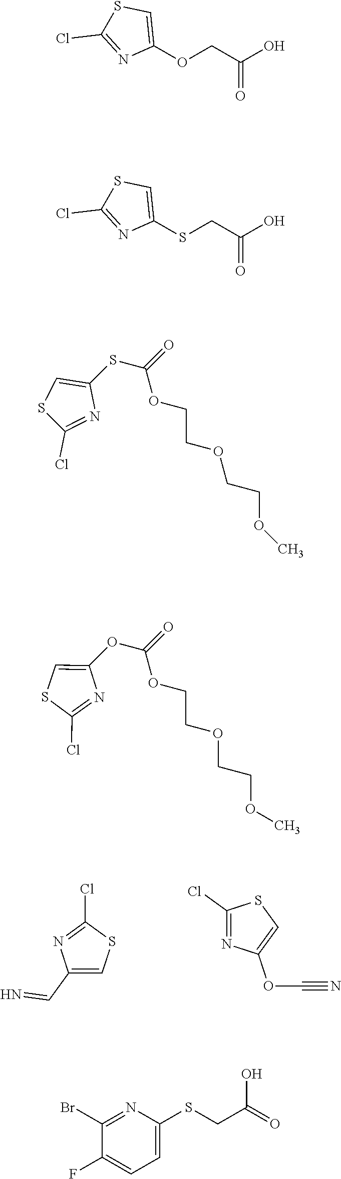 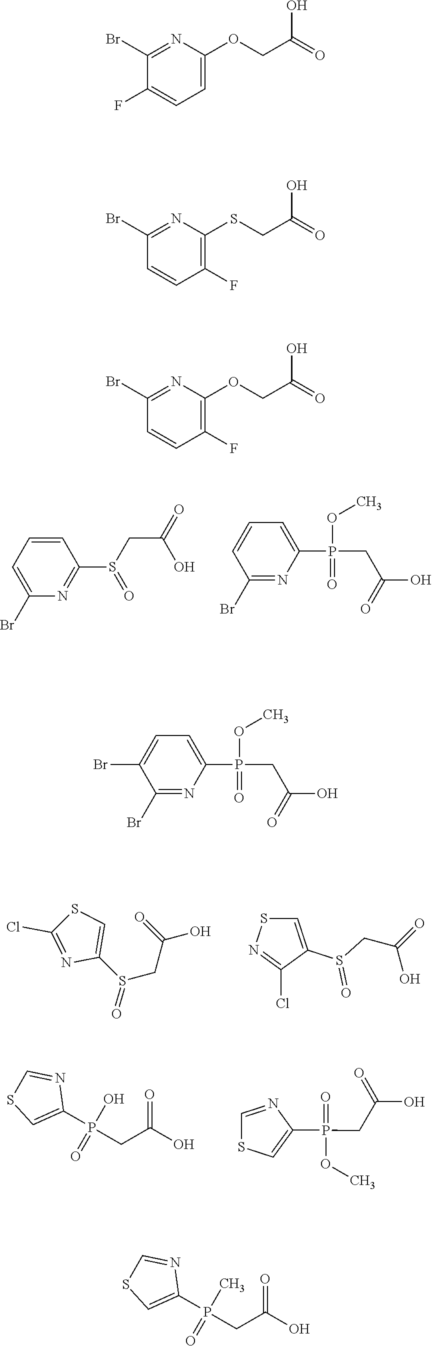
or a salt thereof.

8-9. (canceled)

10. A method of controlling undesired plant growth comprising applying to the locus of said undesired plant growth the herbicidal composition of claim 1.

11. A method of controlling undesired plant growth, comprising applying to the locus of the undesired plant growth a herbicidally effective amount of a compound having the structure 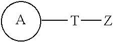
or a salt thereof, wherein:
A is a cyclopentadiene, or benzene scaffold comprising from 1 to 4 heteroatoms; wherein each of the heteroatoms is independently selected from the group consisting of N, S, and Se; and wherein one or more of the carbon, nitrogen, sulfur or selenium atoms of the ring are optionally chemically attached to at least one of —CX₃, —CX₂R, —COX, —CHO, —COR, —CO₂R, —CONH₂, —CONHR, —CON(R)₂, ═S, ═O, —F, —Cl, —Br, and —I, and wherein X is selected from F, Cl, Br and I;
T is —(CH₂)ₙ—, —OR₁—, —SR₁—, —SOR₁—, —CSR₁—, —CS—, —POHR₁, —PO(OR₁)R₁—, —CO—, or —COR₁;
Z is H, substituted or non-substituted alkyl, substituted or non-substituted aryl group, —COOH, —COO—, —OH, —O—R, —COOR with saturated or non-saturated alcohol residues with straight, branched, cyclic, aromatic or heteroaromatic chain; —O—(CH₂CH₂O)ₙR (n≥1), —O—(CHMeCH₂O)ₙR (n≥1)), substituted or unsubstituted phosphate group, carbamoyl group, primary amine, secondary amine, tertiary amine, carboxamide, cyano group, —NR—O—R, —O—NR, or a salt thereof; and,
wherein R is H, substituted or non-substituted alkyl, or substituted or non-substituted aryl group; and, wherein R₁ is —(CH₂)ₙ— (n is 0 to 9).

12. The method of claim 11, further comprising applying to the locus of the undesired plant growth at least one crop protection agent.

13. The method of claim 12, wherein the crop protection agent is selected from the group consisting of herbicides, fungicides, insecticides and plant growth regulators.

14. The method of claim 13, wherein the crop protection agent is amino acid synthesis inhibitor herbicide.

15. The method of claim 14, wherein the amino acid synthesis inhibitor herbicide is selected from the group consisting of imazamox, imazapic, imazethapyr, imazaquin, imazapyr, imazamethabenz, Chlorimuron, Primisulfuron, Thifensulfuron, Triasulfuron, Nicosulfuron, Metsulfuron, Tribenuron, Rimsulfuron, Triflusulfuron, Glyphosate

16-18. (canceled)

19. The method of claim 13, wherein the plant growth regulator is selected from the group consisting of dicamba, 2,4-D, clopyralid and fluroxypyr.

20. The method of claim 12, further comprising applying a third herbicide or a plant growth regulator.

21. The method of claim 11, wherein the locus of the undesired vegetation is field of a crop.

22. The method of claim 21, wherein the crop is selected from the group consisting of maize, wheat, rice, barley, sorghum, and oats.

23. The method of claim 11, wherein the herbicidal composition is applied pre-emergence or post-emergence.

24. (canceled)

25. A method of selectively controlling growth of harmful weeds, comprising applying to a field of a crop an effective amount of the herbicidal composition of claim 1.

26. The method of claim 25, wherein the herbicidal composition is applied pre-emergence or post-emergence.

27. (canceled)

28. The method of claim 25, wherein the crop is selected from the group consisting of maize, wheat, rice, barley, sorghum, and oats.

29. The herbicidal composition of claim 1, wherein T is —(CH₂)ₙ—, —OR₁—, —SR₁—, —SOR₁—, —CSR₁—, —CS—, —POHR₁—, —PO(OR₁)R₁—, —CO—, and —C(O)R₁—, wherein R₁ is —(CH₂)ₙ— (n is 0 to 2).

30. The method of claim 11 wherein T is —(CH₂)ₙ—, —OR₁—, —SR₁—, —SOR₁—, —CSR₁—, —CS—, —POHR₁—, —PO(OR₁)R₁—, —CO—, and —C(O)R₁—, wherein R₁ is —(CH₂)ₙ— (n is 0 to 2).
